(19) United States
(12) Patent Application Publication
(10) Pub. No.: US 2002/0142003 A1
(43) Pub. Date: Oct. 3, 2002

(54) Tumor-associated antigen (B345)

(76) Inventors: Norbert Schweifer, Wien (AT); Marwa Scherl-Mostageer, Wien (AT); Wolfgang Sommergruber, Purkersdorf (AT); Roger Abseher, Wien (AT)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/899,569

(22) Filed: Jul. 6, 2001

Related U.S. Application Data
(60) Provisional application No. 60/243,158, filed on Oct. 25, 2000.
(60) Provisional application No. 60/297,747, filed on Jun. 14, 2001.

(30) Foreign Application Priority Data
Jul. 7, 2000 (DE) DE 100 33 080.0
Apr. 19, 2001 (DE) DE 101 19 294.0

(51) Int. Cl. A61K 39/00; C12Q 1/68; C07H 21/04; C12N 9/00

(52) U.S. Cl. 424/185.1; 435/183; 435/6; 536/23.2

(57) ABSTRACT

Tumour-associated antigen B345 and DNA molecules coding therefor.

The present application claims the benefit, under 35 U.S.C. § 119, of the earlier filing dates of German Patent Application No. DE 100 33 080.0, filed Jul. 7, 2000; German Patent Application No. DE 101 19 294.0, filed Apr. 19, 2001; U.S. Provisional Application No. 60/243,158, filed Oct. 25, 2000; and U.S. Provisional Application No. 60/297,747, filed Jun. 14, 2001. The contents of each of these applications are entirely incorporated herein by reference.

The invention relates to the chemotherapy of tumour diseases.

Normal body cells are subject to a strictly regulated system which controls growth, cell division and the dying off of certain cells. Thus, an adult's body cells divide only when they are replacing dead cells or healing an injury. Cancer cells, on the other hand, carry on growing out of control, they accumulate and form a tumour. When a tumour reaches a critical size, cancer cells may be transported through the bloodstream or lymphatic system into other parts of the body and form colonies there (metastases). Not all tumours are cancerous, as benign tumours do not metastasise and are therefore not usually life-threatening as they can be surgically removed. More detailed information on this subject and the other aspects of tumour formation discussed hereinafter can be found in the following publications: Rauscher and Vogt, 1997; Kastan, 1997; Hesketh, 1995; Pusztai, Lewis and Yap, 1995.

The transformation of a healthy cell into a cancer cell may be triggered by a whole range of factors, such as environmental influences, radiation, viruses or chemical reagents. However, epigenetic modifications (methylations, acetylations and altered chromatin structure) and genetic modifications (point mutation, deletion, amplification, translocation) also play a crucial role in tumour development.

Mutations in coding regions of genes which are involved in regulating cell proliferation may contribute to the conversion of a normal cell into a tumour cell, as the transformed cell has growth advantages over its healthy neighbouring cell.

Cancer is therefore produced by an accumulation of inherited or acquired mutations in critical protooncogenes or tumour suppresser genes.

Cell proliferation is under the control of various gene systems, whereas products of oncogenes are involved in signal transmission from the cell surface to the cell nucleus, cyclin-dependent protein kinase and the inhibitors thereof accompany the cell through the cell cycle. Not infrequently, disorders in the synthesis of these proteins are found in tumour cells. The p53 protein plays a crucial role.

Proteins of the RB protein type regulate the availability of critical transcription factors.

The genes which are highly regulated in tumour tissues are usually the starting point for more detailed analyses and as proteins of all kinds of functions are highly expressed, the approach for therapeutic interventions may take many forms. The objective of cancer research is therefore to find new target molecules (so-called targets) for therapeutic interventions, which can then be used for a targeted therapy with few side effects.

The primary objective is therefore to detect molecular changes between normal tissue and tumour at the level of gene expression (“transcription level”), which should identify new targets, on the one hand, and may be used to develop or find substances for inhibiting malfunctions, on the other hand.

A whole range of different methods of identifying and characterising new targets which form the starting point for developing new therapeutic agents are based on drawing up differential mRNA transcription profiles between tumours and normal tissues. These include differential hybridisation, establishing subtractions cDNA banks (“representational difference analysis”; Hubank and Schatz, 1994; Diatchenko et al., 1996) and the use of DNA chip technology or the SAGE method (Velculescu et al., 1995).

As well as immunotherapeutic approaches, targeted chemotherapy plays an essential role in the treatment of cancer. By chemotherapy is meant the administration of substances which have either a cytostatic or cytotoxic/cytolytic effect as a result of interfering with the metabolism, signal transduction and cell division processes of malignant cells. Chemotherapeutic agents can be divided into various categories on the basis of influencing specific targets in the tumour cell, the type of cellular interaction and interaction with a specific phase of the cell cycle.

The type of cancer treatment depends on the stage of the tumour, the critical point being whether metastases are already present and how far they have spread through the body. The administration of cell toxins for cancer treatment, as well as surgical intervention and radiotherapy, is an integral part of present-day therapeutic concepts in oncology.

Chemotherapy has essentially two main aims: the primary one is to cure cancer; this means that the tumour disappears and does not return. If a cure is no longer possible for various reasons, attempts are made to restrict or control the growth and spread of the tumour.

In principle, substances used in chemotherapy are effective in all dividing cells. Tumour cells, however, are more sensitive to chemotherapeutic agents than, healthy cells, as it is mainly strongly proliferating cells which are attacked.

Every tissue has its own growth characteristics, including cell division, stoppage of growth, differentiation and ageing, which are affected and regulated by internal and external factors. Many of the cytotoxic chemotherapeutic agents currently used are effective only on proliferating cells (not in the G0 phase of cell division). Both normal and cancer cells are attacked. The destruction of normal cells may lead to severe side effects; e.g. destruction of the blood cell-producing tissues of the bone marrow (myelosuppression).

Chemotherapeutic agents are divided into various categories depending on how they affect specific substances within the tumour cell, the cellular processes with which the drugs interact and the phase of the cell cycle which they influence. This information is necessary to oncologists in order to decide which preparations can be combined with one another in the therapy.

The highly regulated genes in tumour tissues are thus potential new target structures and, as proteins of all kinds of functions are highly expressed, this is a very versatile approach for therapeutic interventions.

The goal of cancer research is therefore to find new targets for therapeutic interventions which can then be used for targeted therapy with fewer side effects, compared with the therapeutic agents currently used.

In tumour tissues highly regulated genes are points of attack and hence potential target structures for chemotherapy.

The problem of the present invention was to provide a new protein, preferably expressed by tumour cells, which is a target molecule for intervention using chemotherapeutic methods.

This problem was solved by first producing a cDNA subtraction library, using RDA (“representational difference analysis”) between a lung-adenocarcinoma cell line (A549) and normal lung tissue. In order to select the antigens overexpressed in the tumour the cDNA clones obtained were then sequenced and compared with sequences available in data banks. Among the annotated genes, there were 321 unknown ones, for which there were in most cases ESTs (“expressed sequence tags”)-entries in the data bank. After further qualitative PCR analysis in cDNA libraries of critical normal tissues and immunoprivileged tissues and more detailed data bank searches the number of candidate clones was restricted to 59, the ESTs of which do not come from critical normal tissues.

These clones were spotted onto Incyte DNA chips and hybridised with a whole range of tumour tissues and normal tissues as a reference. The mRNA expression profiles of EST fragments which are differentially, expressed in cancer tissues and normal tissues and belong to an as yet unknown gene were verified using various methods.

The length of the transcripts was determined by Northern blot analysis and the expression pattern in different cell systems was exactly characterised by quantitative PCR. Only unknown genes or ESTs with tumour-specific expression profiles were followed up and subjected to “full length cloning”. Potential ORFs (“open reading frames”) are converted into the corresponding amino acid sequence and analysed for any possible prediction of function by in silico strategies.

Human B345-cDNA was cloned, and the sequence obtained in a first attempt at cloning is shown in SEQ ID NO: 1. Sequence analysis of the human B345-cDNA cloned on this occasion showed a continuous open reading frame from position 215 to position 2461 (excluding stop codon) which, at the nucleotide and protein level, shows no homology or identity to the known sequences of the data banks. The data obtained from the Northern Blot experiments lead one to conclude that the B345 transcript is about 6.5 kb long. In a first attempt, a B345-cDNA with 5897 bp (excluding the polyA region) was obtained as the cloned region, while the presence of a polyadenylation signal and the PolyA tail at the 3′-end of the sequence indicated that the cDNA is complete in this region. Because no continuous reading frame appeared in the 5′ region of the cloned cDNA from position 1 to 214, it was initially assumed that the ATG at position 215, which also corresponds 75% to a Kozak translation initiation site (ACCATGT) (Kozak, 1987), is the start codon of B345.

In another cloning attempt, additional information about the sequence of B345 located further upstream was obtained by a standard method of molecular biology, specifically by so-called “Promotor Finder DNA Walking”.

Thus, the B345 sequence (SEQ ID NO: 1) obtained in the first cloning attempt was expanded in the 5′ region. The start of transcription was located precisely using primer extension analysis and is found at position 201 (SEQ ID NO: 3). By repeated sequencing in the 3′ region too, an additional base was found at position 2430, compared with the sequence shown in SEQ ID NO: 1, which leads to a shift in the reading frame and thus displaces the stop codon from position 2729 to 2791. The cDNA obtained (SEQ ID NO: 3) has an open reading frame which codes for a potential protein with a length of 836 amino acids (SEQ ID NO: 4). The translation initiation site at position 283 corresponds roughly 70% to a Kozak consensus sequence.

The promoter region 200 bp upstream of the presumed transcription start site contains neither a TATA nor a CCAAT box, but does contain a clear GC box, which is a binding site of the SP1 protein. The fact that the GC content in the 5′ region is more than 60% indicates a CpG Island (Bird, 1986).

The resulting primary amino acid sequence of B345 is shown in SEQ ID NO: 4. Analysis of the hydrophilicity plot of the amino acid sequence shows that the B345 protein has two characteristic hydrophobic domains which represent a signal peptide and a helical transmembrane domain (FIG. 6). This polarised structure indicates that B345 is an integral membrane protein.

The extracellular domain leads one to conclude that there is definitely one and possibly three CUB domains. CUB domains occur in various proteins, generally ones which have been regulated during the development of the embryo. A recent publication (Gerstein et al., 2000) demonstrates that proteins containing CUB domains are the most markedly defferentially regulated proteins in C. elegans. Since genes which play a key role in embryonic development perform corresponding functions in cancer, e.g. in cell division, cell proliferation or signal transmission, it can be assumed that overexpression of B345 in cells causes changes in the properties of substrate adhesion or the extracellular matrix. The B345 protein has 12 potential N-glycosylation sites which can be found in the presumed extracellular domain.

In view of its amino acid sequence it can be assumed that the B345 protein constitutes a β-Sheet secondary structure, as it is known that CUB domains fold into a β sandwich.

The intracellular domain (section 691-836) has no significant homologies. However, the whole C-terminus showed 82% identity over 124 amino acids with an EST (Acc No. AW063026) of human ovarian cancer cells.

Starting from the functions of other proteins containing CUB domains it can be concluded that the B345 transmembrane protein plays a part in the communication, interaction and/or the signal transduction with extracellular components or ligands. Moreover, the data of the expression analysis give a strong indication that B345 is implicated in the metastatic process of cancer, particularly cancer of the large intestine.

To clarify the physiological function and role of B345 in metastasis, the following methods of investigation may be used:

First, cell lines, preferably human cell lines, which do not endogenously express B345, were identified,,e.g. using TaqMan PCR. The cells were transfected with a plasmid which contains the B345 sequence and B345 was expressed. Changes in the morphology and/or migration characteristics, found e.g. by soft agar assay (Hamburger and Salmon, 1977) or migration assay (Liaw et al; 1995) of the B345 expressing cells compared with the untransfected cells, indicate that B345 plays a part in the biological process responsible. This is a clear indication of the involvement of B345 in the interaction of tumour cells with one another and/or with the extracellular matrix and hence of a function in metastasis.

Alternatively or in addition to this functional analysis, in a complementary approach, the expression of B345 in cells which express this protein endogenously was suppressed in order to extablish, again, any changes in the morphology or migration characteristics.

In addition, an investigation is optionally carried out to discover whether there are protein components which interact with B345 inter- or extracellularly (e.g. using the Yeast Two Hybrid System (Fields and Song, 1989).

Thus, in a first aspect, the invention relates to a tumour-specific polypeptide designated B345 with the amino acid sequence shown in SEQ ID NO: 4 or a polypeptide which is coded by a polynucleotide which hybridises under stringent conditions with a polynucleotide of the sequence shown in SEQ ID NO: 3 or a partial sequence thereof, as well as protein fragments or peptides derived therefrom.

In another aspect the present invention relates to an isolated DNA molecule coding for the tumour-specific polypeptide designated B345.

Preferably, the DNA molecule according to the invention is a polynucleotide having the sequence shown in SEQ ID NO:3 or a fragment thereof or a DNA molecule which hybridises under stringent conditions with a DNA molecule of the sequence shown in SEQ ID NO: 3 or a partial sequence thereof, or a fragment thereof.

By “stringent conditions” is meant, for example: incubation overnight at 65° C.-68° C. with 6×SSC (1×SSC=150 mM NaCl, 15 mM Tri-sodium citrate), 5×Denhardt's solution, 0.2% SDS, 50 μg/ml salmon sperm DNA, followed by washing twice for 30 min with 2×SSC, 0.1% SDS at 65° C., once for 30 min with 0.2×SSC, 0.1% SDS at 65° C. and optionally finally rinsing with 0.1×SSC, 0.1% SDS at 65° C., or equivalent conditions.

The DNA molecules according to the invention code for (poly)peptides designated B345 with the amino acid sequence shown in SEQ ID NO: 4 or for protein fragments or peptides derived therefrom; thus, DNA molecules or fragments which contain deviations from the sequence shown in SEQ ID NO: 3 as a result of the degeneration of the genetic code are also included.

In one embodiment, the invention relates to an isolated DNA molecule of the sequence shown in SEQ ID NO: 3 or a fragment thereof, or a DNA molecule which hybridises with a DNA molecule having the sequence shown in SEQ ID NO:3 or with a partial sequence thereof, coding for the natural B345 polypeptide or for a fragment thereof.

The B345 DNA molecules may be used in a so-called DNA vaccine for the immunotherapy of tumours.

The B345 DNA molecules according to the invention, preferably in recombinant form as plasmids, may be administered directly or as a component of a recombinant virus or bacterium. In theory, any gene-therapeutical method for the immunotherapy of cancer based on DNA (DNA vaccines) may be used on B345-DNA, both in vivo and ex vivo.

Examples of in vivo administration are the direct injection of “naked” DNA, either by intramuscular route or using a gene gun, which has been shown to lead to the formation of CTLs against tumour antigens. Examples of recombinant organisms are vaccinia virus, adenovirus or listeria monocytogenes (a summary was provided by Coulie, 1997). Moreover, synthetic carriers for nucleic acids such as cationic lipids, microspheres, micropellets or liposomes may be used for in vivo administration of nucleic acid molecules coding for B345 peptide. Different adjuvants which enhance the immune response may also be administered, e.g. cytokines, either in the form of proteins or plasmids coding for them. The application may optionally be combined with physical methods, e.g. electroporation.

An example of ex vivo administration is the transfection of dendritic cells as described by Tuting, 1997, or other APCs which are used as cellular cancer vaccine.

Thus, according to another aspect, the present invention relates to the use of cells which express B345, either per se or, in optionally modified form, after transfection with the corresponding coding sequence, in order to produce a cancer vaccine.

Alternatively to the natural B345-cDNA or fragments thereof, modified derivatives may be used. These include sequences with modifications which code for a protein (fragment) or peptides with greater immunogenicity; the same considerations apply to the modifications at the DNA level as to the peptides described above. Another type of modification is the lining up of numerous sequences, coding for immunologically relevant peptides, in the manner of a string of beads (Toes et al., 1997). The sequences may also be modified by the addition of auxiliary elements, e.g. functions which ensure more efficient release and processing of the immunogen (Wu et al., 1995). For example, by the addition of a locating sequence in the endoplasmatic reticulum (“ER targeting sequence”) the processing and hence the presentation and, lastly, the immunogenicity of the antigen can be increased.

In another aspect the present invention relates to a recombinant DNA molecule which contains B345 DNA, e.g. connected to a regulatory DNA sequence, particularly a heterologous regulatory DNA sequence, e.g. a promoter or enhancer.

In another aspect the present invention relates to antibodies against B345 or fragments thereof. Polyclonal antibodies may be obtained in conventional manner by immunising animals, particularly rabbits, by injecting the B345-antigen or fragments thereof, and then purifying the immunoglobulin.

Monoclonal anti-B345-antibodies may be obtained by standard procedures according to the principle described by Köhler and Milstein, 1975, by immunising animals, particularly mice, then immortalising antibody-producing cells of the immunised animals, e.g. by fusion with myeloma cells, and screening the supernatant of the resulting hybridomas by standard immunological assays for monoclonal anti-B345-antibodies. For therapeutic or diagnostic use in humans these animal antibodies may optionally be chimerised (Neuberger et al., 1984; Boulianne et al., 1984) or humanised (Riechmann et al., 1988; Graziano et al., 1995) in the conventional manner.

Human monoclonal anti-B345-antibodies (fragments) may also be obtained from so-called “Phage Display Libraries” (Winter et al., 1994; Griffiths et al., 1994; Kruif et al., 1995; McGuiness et al., 1996) and using transgenic animals (Bruggemann et al., 1996; Jakobovits et al., 1995).

The anti-B345-antibodies according to the invention may be used in immunohistochemical analyses for diagnostic purposes, or as therapeutic agents in cancer therapy. (One example of the successful use of a monoclonal antibody in cancer therapy is herceptin; an antibody against the proto-oncogene HER2. Herceptin can be used in breast cancer patients exhibiting an overexpression of HER2.)

According to another aspect the invention relates to the use of B345-specific antibodies for selectively delivering any desired substances to or into a tumour which expresses B345. Examples of such substances are cytotoxic agents or radioactive nuclides, the effect of which is to damage the area surrounding the tumour. Because of the relatively tumour-specific expression of B345 only mild side effects are to be expected. In another aspect substances for visualising tumours which express B345 may be used with the aid of B345-antibodies. This is useful for the diagnosis and for evaluating the course of therapy. Therapeutic and diagnostic uses for antibodies which may be used as anti-B345 antibodies are described in WO 95/33771.

The protein designated B345 according to the present invention and the protein fragments, peptides or peptide equivalents or peptidomimetics derived therefrom may be used in cancer therapy, e.g. to induce an immune response to tumour cells which express the corresponding antigen determinants. They are preferably used for the treatment of B345-positive tumours, particularly in lung and colon carcinoma.

B345 or peptides, peptide equivalents and peptidomimetics can be used for the immunotherapy of cancer, as described e.g. in WO 00/73438, to the disclosure of which reference is hereby made.

It is known that tumour-associated antigens may have tumour-specific mutations which contribute to an immunological differentiation between tumour and normal tissue (Mandruzzato et al., 1997; Hogan et al., 1998; Gaudi et al., 1999; Wölfel et al., 1994). In order to detect the presence of tumour-specific B345 mutations, appropriately using probes of the isolated cDNA according to the invention, the B345-cDNA from one or more different tumours is cloned and the sequences obtained are compared with normal tissue-B345-cDNA. Tests are carried out which are intended to show whether tumour-B345 peptides from a section of sequence which is mutated compared with normal tissue-B345 have greater immunogenicity than normal tissue-B345 peptides from the corresponding section. To confirm that some mutations are tumour-specific, antibodies against these regions may be generated and tumour cells may be investigated for the expression of possible mutations.

Thus, in another aspect, the present invention relates to B345-peptides, derived from regions of a tumour-expressed B345 which contain tumour-specific mutations.

It can be assumed, from the preferred expression of B345 in tumour cells, that this protein has an important function for the tumour, e.g. for its formation, infiltration and growth and thus constitutes a target for chemotherapeutic intervention.

With a view to its use as a target in targeted chemotherapy B345 is characterised in more detail so as to develop a suitable strategy for intervention with this function.

As the first step in the so-called “down-stream” functional analysis of B345, a bioinformatic analysis is conveniently carried out in a first step, to indicate the direction for the experimental validation of B345 as target.

For this analysis the bioinformatic concepts based on similarity and modular structure form an essential basis. Established bioinformatic tools for detecting similarities are BLAST (http://www.ncbi.nlm.nih.gov/BLAST, Altschul et al., 1997) or FASTA (Pearson & Lipman, 1988), the specialised data banks such as Pfam (http://www.sanger.ac.uk/Pfam, Bateman et al., 2000) and SMART (http://smart.embl-heidelberg.de, Schultz et al., 2000), which take account of domain structures. To refine the analysis applications such as Clustal (http://www2.ebi.ac.uk/clustalw, Higgins et al., 1996) HMMer (http://hmmer.wustl.edu), PSI-BLAST (Altschul et al., 1997) and the PROSITE data bank (http://www.expasy.ch/prosite, Hofmann et al., 1999) may be used. Statistical methods of analysis which are not based on homologies make it possible to predict other structurally and functionally relevant properties such as the secondary structure and the occurrence of transmembrane segments and helix-turn-helix motifs. There are methods of predicting the secondary structure of proteins; Jpred (http://barton.ebi.ac.uk/servers/jpred.html, Cuff et al., 1998) is particularly worth mentioning. Predicting the secondary structure can underpin functional hypotheses, e.g. if the structure of the suspected homologue is known.

According to bioinformatic analysis B345 has a helical transmembrane domain, both the N-terminal and the C-terminal region being hydrophilic, leading to the conclusion that this protein is a transmembrane protein. The N-terminal, extracellular region has a few CUB domains which have a tendency to form disulphide bridges and are therefore involved in dimerisation or protein-protein interactions (Bork et al., 1993). The C-terminal, intracellular end shows homology with a receptor kinase and a C-kinase substrate.

B345 is subsequently subjected to biochemical and biological analysis.

In a subsequent step the function of B345 for the progress of the tumour is clarified; e.g. by proliferation assays in vitro or in animal models which overexpress the B345 gene under investigation (constitutively or inducibly) and as a control either express it in deleted (inactive) form or down-regulate it using antisense (cf. e.g. Grosveld and Kollias, 1992).

B345 may be used in screening assays to identify substances which modulate, particularly inhibit, the activity of this protein. In one embodiment an assay of this kind may consist, for example of introducing the B345 protein, or an active fragment thereof, into cells which react to the activity of B345 with proliferation or of expressing the corresponding B345 cDNA in the cell, and determining the proliferation of the cells in the presence and absence of a test substance.

One example of test cells might be cells with a low division rate, e.g. primary cells containing no endogenous B345. To determine the suitability of the cells for a screening assay, the cells are transformed with B345-cDNA, cultured and tested using standard assays, e.g. the incorporation of thymidine, for their ability to proliferate. On the basis of a significant increase in their proliferation after B345 expression they may be used as test cells, e.g. in High Throughput Screening Proliferation assays. Examples of proliferation assays in the High Throughput format, e.g. based on the MTS assay, are described in WO 98/00713.

Substances with a proliferation-inhibiting effect may be used to treat tumours with a significant B345 expression, particularly in carcinoma of the lung and colon.

### SUMMARY OF THE FIGURES

FIG. 1A: expression profile of B345, B452 and B540 in individual lung carcinomas and lung tumour cell lines.

FIG. 1B: expression profile of B345 in normal large intestine tissue and tumour cell lines.

FIG. 1C: graphic representation of the alignment of B345, B452 and B540.

FIG. 2A: Northern Blot analysis of the tumour cell line A549 using a 490 bp long B345 PCR product

FIG. 2B: Northern Blot analysis of various normal tissues using a 490 bp long B345 PCR product

FIG. 2C: Northern Blot analysis of various cancer tissues using a 318 bp long B345 PCR product

FIG. 3 mRNA expression analysis of B345 by real-time PCR of tumour and normal tissues.

FIG. 4: mRNA expression analysis of B345 by real-time PCR of laser-microscope-prepared large bowel tumours (LCM) and normal large bowel tissue and tumour cell lines.

FIG. 5: Graphic representation of the gene structure of B345.

FIG. 6: Hydrophilicity and transmembrane blot of the B345 protein

FIG. 7: Potential protein structure of B345

### DESCRIPTION OF THE TABLES

Tab. 1: Summary of the Northern Blot data for B345 in various normal tissues (1A), cancer cell lines (1B); and various normal tissues compared with the corresponding tumour tissue (1C)

Tab. 2A: Summary of the data for the quantitative PCR of B345 in various normal and cancer tissues

Tab. 2B: Summary of the data for the quantitative PCR of B345 in various normal tissues and microdissected colon adenocarcinoma tissues

Explanation of Symbols

+++ extremely positive

++ strongly positive

+ positive

(+) slightly positive

− negative

### EXAMPLE 1

RDA (“Representational Difference Analysis”) of the human adenocarcinoma cell line of the lung (A549) and normal lung tissue.

The human lung adenocarcinoma cell line A549 (CCL 185) obtained from ATCC was cultured in T150 cell culture flasks. The nutrient medium used was MEM with 10% beat-inactivated, foetal calf serum and 2 mM of L-glutamine. Every 3 to 4 days the cells were split for propagation by trypsinisation 1:5 to 1:10. After about 80% confluence had been achieved, 4 ml of a trypsin solution (contents per litre: 8 g NaCl, 0.2 g KCl, 1.13 g anhydrous Na₂HPO₄, 0.2 g KH₂PO₄, 100 ml of 2.5% trypsin solution, 1 g EDTA-Na-salt; pH 7.2-7.4) were used per T150 cell culture flask to harvest the cells. The 4 ml were transferred into a 15 ml Falcon test tube, mixed with 8 ml of PBS, centrifuged at 1200 rpm in a Heraeus bench centrifuge (Megafuge 2.0 R) for 5 min at 4° C., the cell pellet was mixed with 1 ml of lysing buffer (10 mM Tris-HCl pH8, 140 mM NaCl, 1.5 mM MgCl₂, 0.5% NP40), shaken vigorously and centrifuged off in a 2 ml Eppendorf vessel at 12,000 rpm and at 4° C. for 5 min in a Sigma bench centrifuge (Sigma 202 MK). The supernatant was transferred into a new Eppendorf vessel and after the addition of 55 μl of 20% SDS solution extracted twice with double the volume of a CHCl₃/phenol (1:1 v/v) mixture and extracted once with a single volume of CHCl₃. The aqueous RNA-containing phase was mixed with 1/10 volume of 3M NaAc (pH5) and twice the volume of 96% EtOH and the RNA was precipitated overnight at −20° C. Starting from 1 mg of total RNA, the procedure for isolating poly-A(+)RNA using the polyATtract Kit (Promega) was carried out according to the manufacturer's instructions. The A549 poly-A(+)RNA was stored in a concentration of 1 mg/ml in DEPC-treated H₂O in aliquots at −80° C.

In order to carry out representational difference analysis (RDA; Hubank and Schatz, 1994; Diatchenko et al., 1996) the poly-A(+)RNA of the lung adenocarcinoma cell line A549 was used as the “tester” and that of normal lung tissue (1 mg/ml; Clontech, Palo Alto; #6524-1) was used as the “driver”. The RDA was carried out using the PCR-select™ kit (Clontech, Palo Alto) in accordance with the manufacturer's instructions, except that a modified primer/adapter-2-oligonucleotide system was used: adapter-2-alt-1 (SEQ ID NO: 31) and nested-PCR-primer-2-alt (SEQ ID NO: 32) and adapter-2-alt-2 (SEQ ID NO: 33). The newly generated primer/adapter sequences make it possible to excise the particular cDNA fragments subsequently, thanks to the presence of three new restriction enzyme cutting sites (Kpn I, Sac I and Xho I) in the sequence of the nested-PCR-primer-2-alt after the subtracted cDNA fragments have been cloned into the pPCRII vector. It was necessary to design a primer/adapter sequence with a plurality of available restriction enzyme cutting sites because point mutations could often be observed, caused by the PCR amplification steps, in the primer sequences in particular.

After the synthesis of double-stranded cDNA using oligo-dT, the cDNA of “tester” and “driver” obtained was digested with RsaI (RsaI is a restriction enzyme which recognises 4 bases and on a statistical average yields CDNA fragments which are 256 bp long). Equal parts of “tester-cDNA” were ligated with either adapter 1 or 2 and then separately hybridised with an excess of “driver-cDNA” at 65° C. Then the two mixtures were combined and subjected to a second hybridisation with fresh denatured “driver cDNA”. The concentrated “tester ”-specific cDNAs were then exponentially amplified by PCR, with primers specific to the adapters 1 and 2. To achieve further concentration, one aliquot from this reaction was subjected to a second PCR with specific nested primers. The exponentially amplified cDNA fragments resulting from this reaction were ligated directly into the pCRII vector (Invitrogen; “TA-cloning vector”) and then one third of the ligation mixture was transfected into competent E. coli (OneShot™, Invitrogen).

712 positive transformants (blue-white selection) were obtained and cultivated in 96-well blocks in LB-Amp Medium (1.3 ml per well) for 48 h at 37° C. 750 μl of the E. coli suspensions were used per well for the preparation of the plasmid-DNA (96-well QIAgen minipreparation method according to the manufacturer's instructions). The remaining bacterial cultures were stored as glycerol stock cultures at −80° C.

A cDNA subtraction library consisting of 712 individual clones was obtained, in the form of both E. coli glycerol stock cultures and also purified plasmids.

### EXAMPLE 2

DNA-sequencing and annotation of TAA candidates

The isolated plasmid-DNA of all the 712 clones (cf. Example 1) was sequenced by the Sanger method on an ABI-377 Prism apparatus. The sequences obtained were annotated using BioScout-Software (LION, Heidelberg) and subjected to data bank comparisons (Genbank). Of 712 clones, 678 were able to be sequenced and annotated. The rest (34) either only had poly(A) sequences as an insert or corresponded to a religated vector or could not be sequenced. Of the 678 annotatable sequences, 357 proved to be genes with a known function. The remaining 321 represented clones coding for genes with an unknown function; 59 of them did not even have entries in the human EST data bank. Known genes were not treated further. For those unknown genes for which an EST entry was available, the expression profile was evaluated: all those ESTs with >95% identity (BLAST) which belonged to the experimentally determined sequence of the subtraction libraries were examined. During annotation the material was subdivided into a) critical normal tissue, b) foetal, “disposable” and immunoprivileged tissue and c) tumours and tumour cell lines. On the basis of this “virtual mRNA profile” (“virtual Northern blot”) 200 clones for which no ESTs were found in group a) were selected for further experimental analyses (including the 59 clones for which there was no EST entry). To narrow down the candidate clones still further, from the sequences determined from the 200 selected clones, pairs of oligonucleotide primers were designed and synthesised. First, 8 different cDNA libraries derived from human tissue (GibcoBRL “SUPERSCRIPT™”), which are directionally cloned in pCMV-SPORT, were tested by qualitative PCR for the presence of the particular candidates. The CDNA libraries used came from heart tissue (#10419-018), liver (#10422-012), Leukocytes (#10421-022), kidney (#10420-016), lung (#10424-018), testis (#10426-013), brain (#10418-010) and foetal brain (#10662-013). The PCR conditions were as follows: 20 μl of total volume per PCR mixture contained 1× TaqPol buffer(50 mM KCl, 10 mM Tris-HCl pH 9, 0.1% Triton X-100), 1.5 mM MgCl₂, 0.2 mM dNTPs (Promega), 0.025 U/μl Taq-DNA-polymerase (Promega), 5 pM each of specific oligonucleotide primer for B345 (B345-D, SEQ ID NO: 34) and (B345-U, SEQ ID NO: 35) and 100 ng of the plasmid-DNA under investigation. Specific primers for GAPDH (SEQ ID NO: 36 and 37) were used as a control. To check the selective detection, the relevant B345 specific primer pairs, oligonucleotide primer (SEQ ID NO: 34) and (SEQ ID NO: 35), were tested in parallel for the isolated plasmid with the original B345 fragment (fragment of B345 CDNA originally isolated). The detectability of fragments of the expected length with a strong signal in one of the critical normal tissues (heart, liver, lung, kidney and leukocytes), but not in the cDNA libraries of immunoprivileged tissues (brain, foetal brain and testis) under these PCR conditions (1 cycle: 3′ 94° C.; 35 cycles: 1′ 94° C.-1′ 55° C.-1′ 72° C.; 1 cycle: 7′ 72° C.) was defined as the distinguishing criterion. Using this qualitative PCR analysis the number of candidates could be reduced to 56; clone B345 was in this already preselected group of candidates.

### EXAMPLE 3

Expression analysis by cDNA chip hybridisation

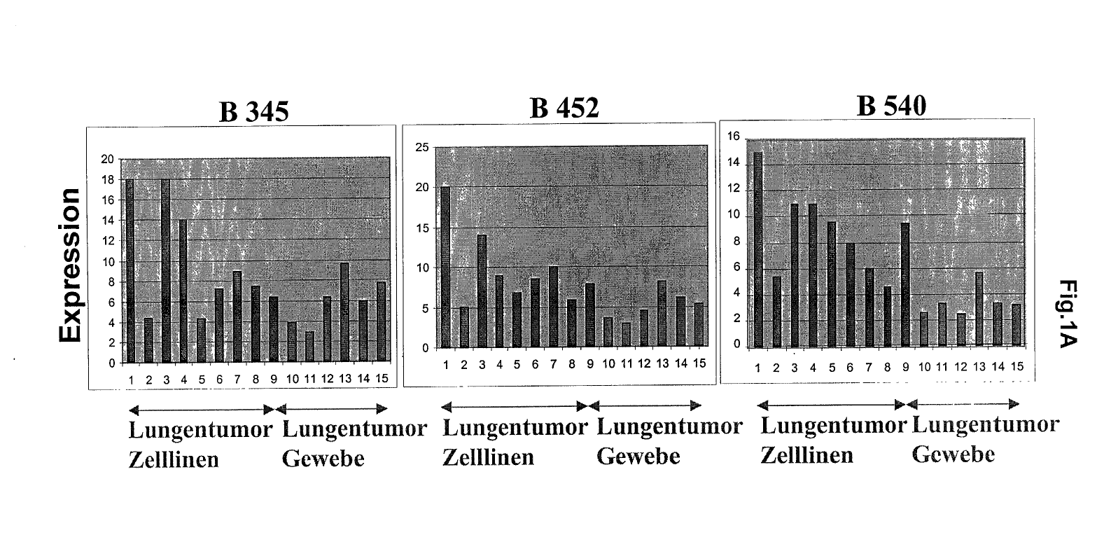

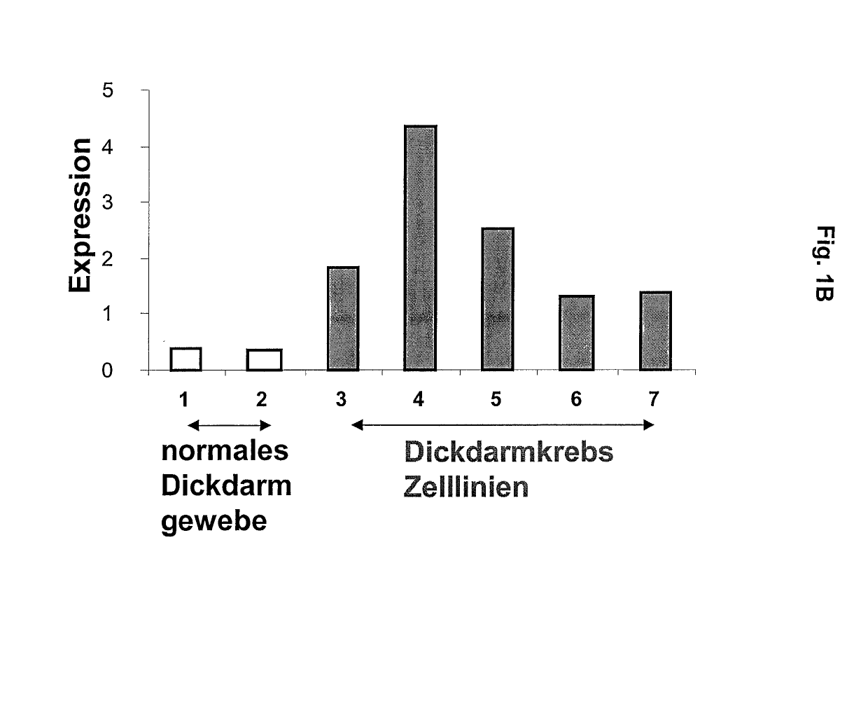

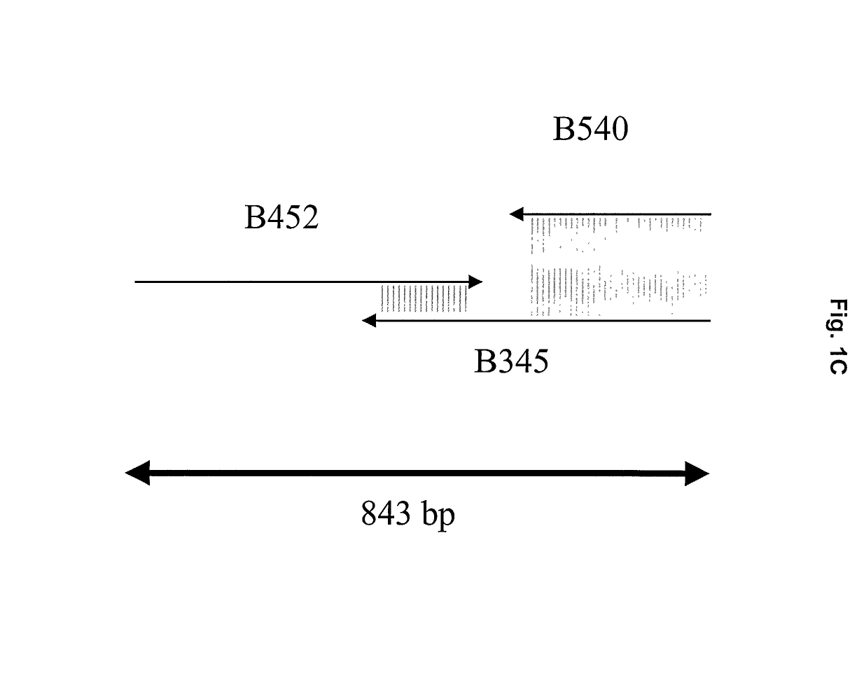

In order to design a cDNA chip a number of clones from categories having all kinds of functions ranging from apoptosis to cell cycle regulation were selected from the dBEST data bank by a nucleotide sequence search. In all, 1299 IMAGE clones were obtained (of which 1024 are known genes) and sequenced to check them. Microtitre plates with bacteria which contain approximately 800 bp long sequences from the 3′ end of the gene in the vector were sent to Incyte Pharmaceuticals, Inc. (USA), where they were spotted onto 60 chips. In addition to these clones, 120 EST clones identified by RDA were also spotted onto the chips. The DNA chips thus produced were then hybridised with Cy3-labelled cDNA from normal tissue, tumour tissue and cell lines together with Cy5-labelled cDNA from a mixture of nine different normal tissues and the two signals were compared in order to standardise the expression values. The calculations were partly carried out in S-Plus or in Microsoft Excel. Evaluation of the chip experiments produced a very similar expression profile for B345, B540 and B452 when hybridisation was carried out with lung cancer probes of cell lines and patient material (cf. FIG. 1A). A tumour-associated expression profile of this kind could also be found for B345 when colon adenocarcinoma was compared with normal colon tissue (cf. FIG. 1B).

The sequence alignment of B345, B540 and B452 clearly showed an overlap between the individual EST fragments. It could therefore be assumed that the three clones are ESTs of one and the same gene. The resulting DNA section covers a length of 843 bp (cf. FIG. 1C) and was used in further experiments to search public data banks. The search results shows no significant homology with known DNA or protein sequences, indicating that B345 is a hitherto unknown gene.

### EXAMPLE 4

Expression analysis of B345 using Northern Blots:

B345 is a gene which is highly regulated in tumour tissues (cf. FIG. 1A and 1B, Tab. 1a and Tab. 1B) according to DNA Chip analyses.

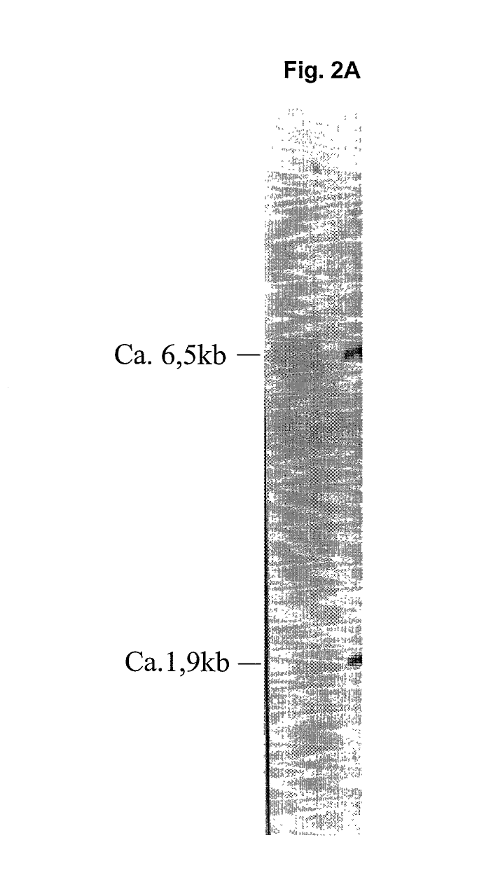

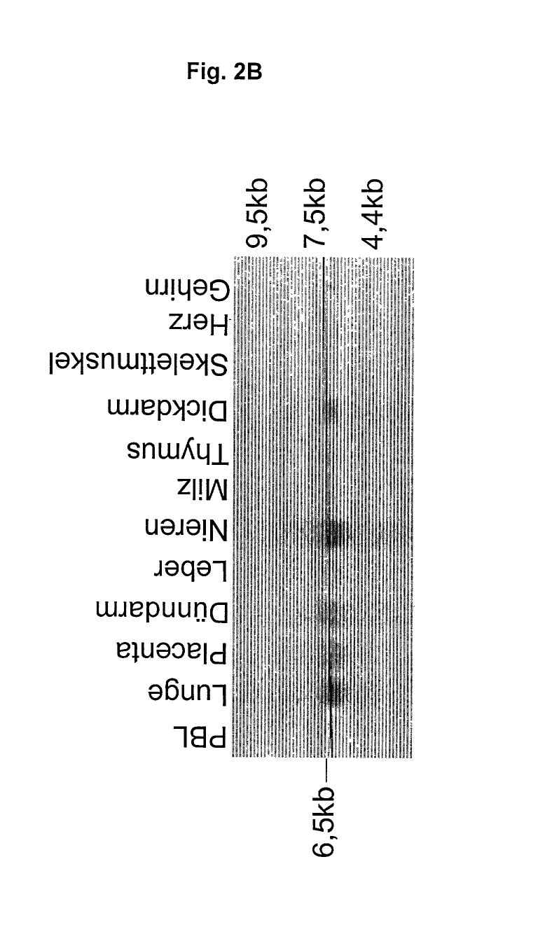

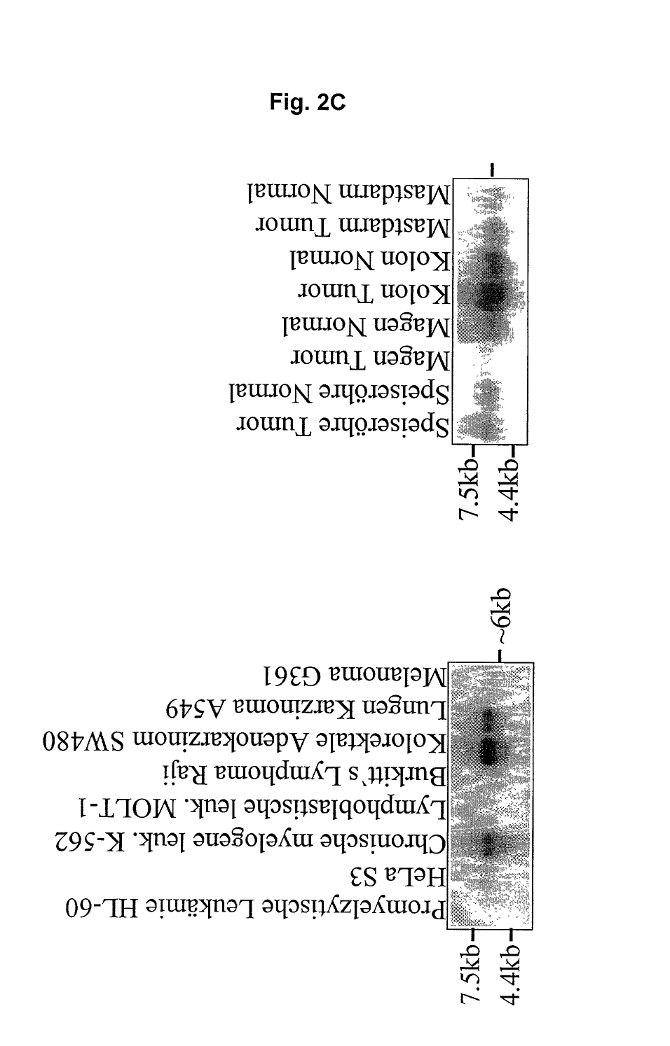

In order to confer the transcription profile obtained, on the one hand, and determine the length of the expected mRNA for full size cloning, on the other hand, a Northern Blot analysis was carried out for B345 using human cell lines and the “Human Multiple Tissue Northern Blots” (Clontech and Invitrogen). The probes used were 490 bp and 318 bp long PCR products of B345 (primer (SEQ ID NO: 5 and SEQ ID NO: 6 or SEQ ID NO: 7 and SEQ ID NO: 8)) labelled with [α-³²P]dCTP (NEN, Boston). The hybridisation took place at 68° for 2′ h; visualisation by standard autoradiography (Hyperfilm, Amersham). FIG. 2A, 2B and 2C and Tab. 1a and Tab. 1B and 1C show the results of this analysis: FIG. 2A the analysis of the cell line A549, FIG. 2B the analysis of 12 normal tissues (Peripheral Blood Lymphocytes (PBL), lung, placenta, small intestine, liver, kidney, spleen, thymus, colon, skeletal muscle, heart and brain) and FIG. 2C the analysis of 8 cancer cell lines (promyelocytic leukaemia HL60, HeLa-S3, chronic myelogenic leukaemia K-562, lymphoblastic leukaemia MOLT-4, Burkitt 's lymphoma (Raji), colon adenocarcinoma SW480, lung adenocarcinoma A549 and melanoma G361). The B345 transcript is 6.5 kb long.

### EXAMPLE 5

Analysis of the expression profile of B345 at the RNA level using quantitative RT-PCR (real time PCR or TaqMan analysis).

In order to quantify more precisely the expression of mRNA in the various normal and tumour tissues, “real time PCR” was used, which makes it possible to calculate the RNA concentration compared with an external standard.

The RNA was isolated from frozen tissue with Trizol according to the instructions provided by the manufacturer, Gibco. To eliminate any contaminating DNA the prepared RNA was digested with DNAase I as follows: 3 μg of total RNA were incubated for 15 minutes at 37° C. with 20 μl of 5× AMV buffer (Promega), 1 μl of RNasin (Promega) and 2 μl of DNase I (Boehringer Mannheim) in a total volume of 80 μl. 120 μl of phenol:chloroform:isoamyl alcohol (25:24:1) were added, mixed in a vortexer and briefly centrifuged. The aqueous phase was removed, 120 μl of chloroform:isoamyl alcohol (24:1) were added and the mixture was centrifuged as before. The purified RNA was precipitated with ethanol and dissolved in water.

Then the total RNA was transcribed into cDNA with reverse transcriptase (Superscript, Gibco, BRL): 1 μl of oligo dT primer (Promega) was added to 3 μg of total RNA and made up to a final volume of 10 μl with water. After 5 minutes′ incubation at 70° C. the solution was cooled for 5 minutes at room temperature. 5 μl of RT reaction buffer (5×, Gibco, BRL), 2.5 μl of dNTPs (10 mM of each, Boehringer Mannheim), 1 μl of RNas in (10 U/μl, Promega), 1.5 μl of Superscript (10 U/μl, Gibco, BRL) and 5 μl of water were added and the mixture was incubated for 1 hour at 42° C. and the reaction was stopped by incubating for 3 minutes at 95° C.

In order to prepare a cDNA pool of a specific type of tissue or tumour, 3 to 10 different individual preparations from different patients were mixed together in equal proportions. The quantitative measurement of the “household genes” β-actin, GAPDH and tubulin in CDNA pools was carried out as follows:

A) β-Actin-TaqMan PCR (Perkin Elmer)

For details of the principle of the TaqMan method cf. manufacturer's information (Perkin Elmer). A TaqMan PCR run contained samples of β-actin control sequence with 10², 10³, 10⁴, 10⁵ and 10⁶ copies/μl (Perkin Elmer) to determine the standard curve, a negative control without DNA and the cDNA pools which are to be quantified. All the samples were analysed in triplicate. For a 25 μl reaction mixture, 1 μl of cDNA, 2.5 μl of 10× buffer A (Perkin Elmer), 4 μl of MgCl₂ (25 mM, (Perkin Elmer)), 0.5 μl of each nucleotide (10 mM of dATP, dCTP, dGTP; 20 mM of dUTP), 0.125 μl of TaqMan probe (20 μM; TaqMan probe for B-actin (SEQ-ID NO: 20 fluorescence-labelled with 6-carboxyfluorescein at the 5′ end and with 6-carboxytetramethylrhodamine at the 3′ end), 1 μl of each β-actin-specific primer (20 μM each of Forward primer SEQ-ID NO: 21 and Reverse primer SEQ-ID NO: 22), 0.25 μl of AmpErase uracil N-glycosylase “UNG” (1 U/μl, Perkin Elmer), and 0.125 μl of AmpliTaq Gold (5 U/μl, Perkin Elmer) were mixed together, transferred into MicroAmp Optical Tubes (Perkin Elmer) and sealed with MicroAmp Optical Caps. The PCR was carried out as follows: one cycle of 2 minutes at 50° C. for the UNG reaction, one cycle of 10 minutes at 95° C. to activate the AmpliTaq, 40 cycles each of 15 seconds at 95° and 1 minute at 60° C. Then the probes were kept at 25° C. The data was evaluated using the “Sequence Detection System 1.5b1” programme (PE Applied Biosystems), basically by comparing the fluorescence signals of the cDNA probes being quantified with the signals of the control plasmid dilutions of known concentration.

B) GAPDH-TaqMan PCR

For quantifying GAPDH, which was used like β-actin or tubulin to standardise the RNAs used, the following primers or probes were used. The TaqMan probe used for GAPDH was a probe (SEQ-ID NO: 23) labelled at the 5′ end with tetrachlorofluorescein and at the 3′ end with carboxymethylrhodamine (Forward GAPDH primer: SEQ-ID NO: 24 and Reverse primer: SEQ-ID NO: 25). The reactions were carried out as described above.

C) Tubulin-SybrGreen PCR (Perkin Elmer)

For the principle of SybrGreen PCR cf. the manufacturer's information (Perkin Elmer). A SybrGreen PCR run contained samples of tubulin control plasmid with 10², 10³, 10⁴, 10⁵ and 10⁶ copies/μl (Perkin Elmer) to determine the standard curve, a negative control without DNA and the cDNA pools which are to be quantified. All the samples were analysed in triplicate. For a 25 μl reaction mixture, 1 μl of cDNA, 2.5 μl of 10× SybrGreen buffer (Perkin Elmer), 3,5 μl MgCl₂ (25 mM, Perkin Elmer) ), 0.5 μl of each primer (20 μM each, Perkin Elmer, Tubulin Forward (SEQ-ID NO:26); Tubulin reverse (SEQ-ID NO: 27), 0.25 μl of AmpErase uracil N-glycosylase “UNG” (1 U/μl, Perkin Elmer), and 0.25 μl of AmpliTaq Gold (5 U/μl, Perkin Elmer) were mixed together, transferred into MicroAmp Optical Tubes (Perkin Elmer) and sealed with MicroAmp Optical Caps. The PCR was carried out as follows: one cycle of 2 minutes at 50° C. for the UNG reaction, one cycle of 10 minutes at 95° C. to activate the AmpliTaq, 40 cycles each of 15 seconds at 95° C. and 1 minute at 60° C. Then the probes were kept at 25° C. The data was evaluated using the “Sequence Detection System 1.5b1” programme (PE Applied Biosystems), basically by comparing the fluorescence signals of the cDNA probes being quantified with the signals of the control plasmid dilutions of known concentration.

D) B345-TaqMan PCR

The quantitative TaqMan-PCR analysis of B345 was carried out as described for the “household genes”. However, B345 specific primers (SEQ ID NO: 28 and SEQ ID NO: 29) (200 ng/μl) and a B345 specific probe (SEQ ID NO: 30, 20 μM) labelled at the 5′ end with tetrachlorofluorescein and at the 3′ end with carboxymethylrhodamine were used. The PCR product of B345 with the primers SEQ ID NO: 28 and SEQ ID NO: 29 with a known copy number was used as the standard.

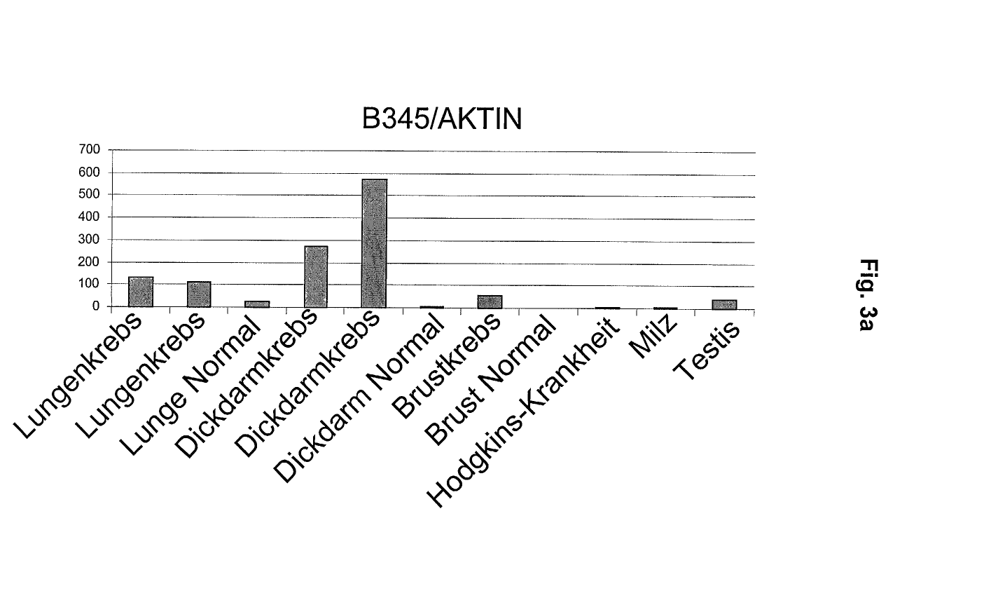

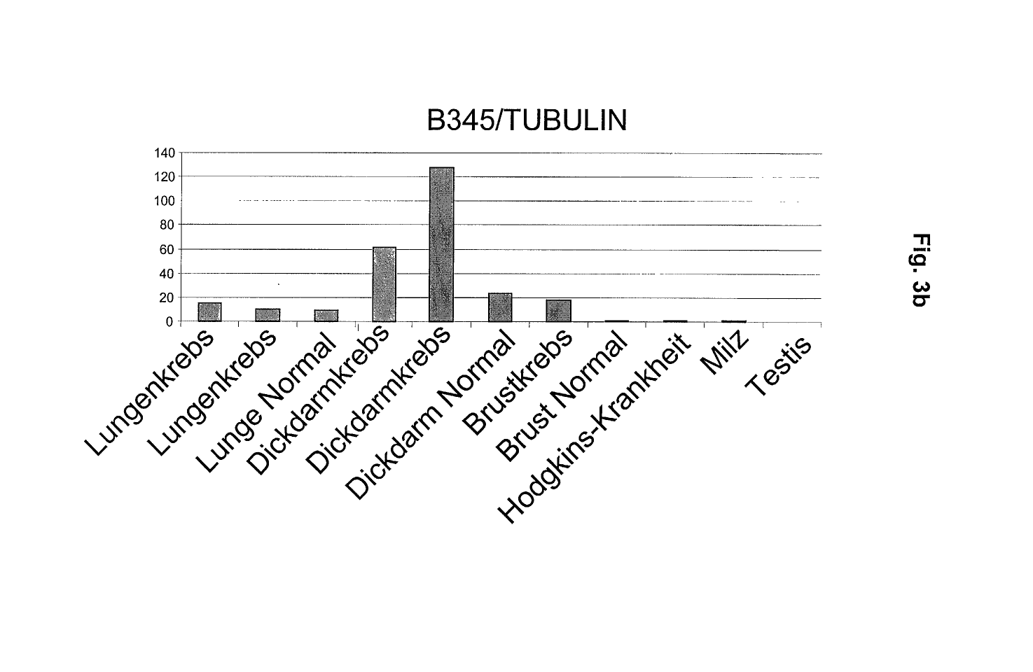

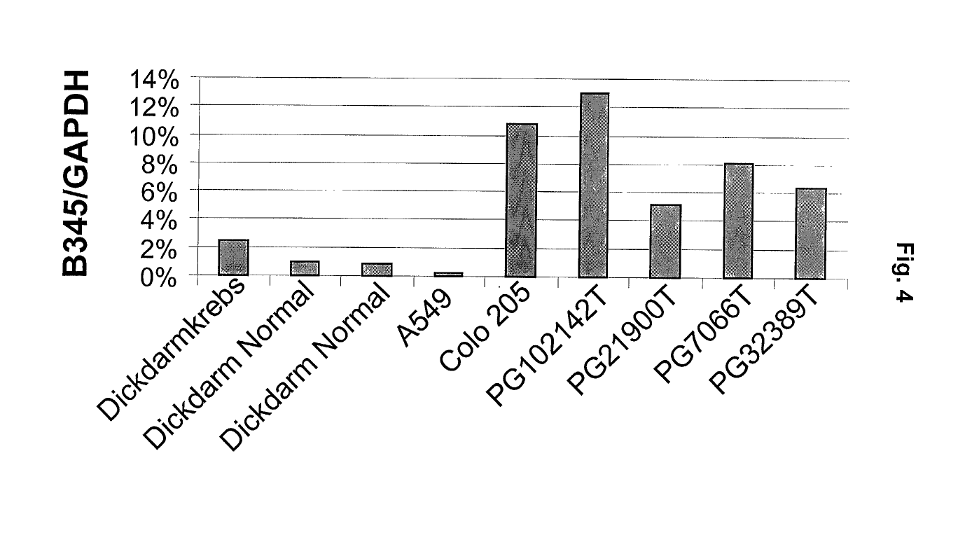

FIG. 3 shows the TaqMan expression analysis (FIG. 3A; β-actin; FIG. 3B: tubulin). It was found that B345 is expressed more highly in large bowel cancer tissue than in normal tissue (cf. Tab. 2a). However, both the normal tissue and the tumour tissue constitute a very heterogeneous mixture of different cell types. Furthermore, the proportion of tumour cells in the tumour tissue varies considerably from about 30 to 80%. In order to minimise this biological heterogeneity, the epithelial cells of the large intestine, which are the cells of origin of adenocarcinoma, and cancer cells or cancer regions were specifically prepared by laser microdissection. Tissue sections 10 μm thick were prepared using a Leica, Jung CM1800 cryomicrotome and placed on a polyethylene-coated slide (Böhm et al., 1997). The sections, dried at ambient temperature for about 30 minutes, were incubated with Mayers haematoxylin (SIGMA DIAGNOSTICS) and then washed under running water for five minutes to remove any non-specifically bound dye. After 5 minutes, drying at 37° C., the laser microdissection was carried out. This was done using the PALM laser microscope (PALM GmbH, Bernried, Germany) and about 2000 to 5000 cells were prepared. The cDNA obtained by Reverse Transcription was analysed by Real Time PCR here too. The results show that the B345 expression in large intestine carcinoma cell lines and in patient material is comparatively many times higher than that of the normal tissue of the large intestine. For standardisation, the expression level of GAPDH was determined (cf. FIG. 4 and Tab. 2B).

### EXAMPLE 6

a) Cloning of the cDNA of B345

Searching through data banks for sequences of gene fragments (ESTs, expressed sequence tags) which can be used for the “in silico” cloning of B345 yielded an overlapping EST contig of about 1500 bp. The polyA region at one of the ends indicated the orientation of the DNA section in relation to 5′-3′ orientation; which is essential when designing new primers for the amplification of B345-specific cDNA fragments.

First, the potential 3′ end described by the data bank analysis was verified by experimental approaches. RNA from the lung carcinoma cell line Calu 6 (AACC No. HTB56) was reverse transcribed using the primer (SEQ ID NO: 9) and the resulting single-strand cDNA was amplified by PCR with the gene-specific primer SEQ ID NO: 5 and the adaptor primer SEQ ID NO: 10.

For a 25 μl PCR mixture, 1 μl of the cDNA pool was mixed in water with 2.5 μl of 10×Taq buffer (Promega), 1.5 μl of MgCl₂ (25 mM, Promega), 0.5 μl of dNTPs (10 mM each, Boehringer Mannheim), 1 μl of primer mixture (20 μM each), 0.15 μl of Taq polymerase (Promega). The PCR was carried out as follows: 1× 94° C. for 3 minutes; 30× 94° C. for 30 seconds, 55° C. for 30 seconds, 72° C. for 1 minute; kept at 4° C. The PCR was analysed on a 1.2% agarose gel.

The two primers were then used to sequence the purified PCR product. The sequences found showed high homology with the DNA fragment cloned “in silico” (including the polyA tract).

Since the cloning of 5′ terminal sequences is usually a very laborious process, various methods were used below to solve the problem.

Here again, Calu 6 was used as the starting cell line. After reverse transcription of the RNA with the primer SEQ ID NO: 9 and synthesis of the second strand, a linker consisting of the two oligos SEQ ID NO: 11 and SEQ ID NO: 12 was ligated onto the double stranded cDNA (Abe et al., 1992). The resulting LoneLinker cDNA library was then amplified with the gene-specific primer SEQ ID NO: 6 in linear manner over 35 cycles. One aliquot of the B345-enriched cDNA could then be further amplified with the primers SEQ ID NO: 13 and LLEcoRIA SEQ ID NO: 11. After gel electrophoresis of one aliquot and Southern analysis with the gene-specific oligo SEQ ID NO: 14, it was possible to locate a 5 kb band. This fragment was then sequenced step by step and aligned with the sequence of the EST contig.

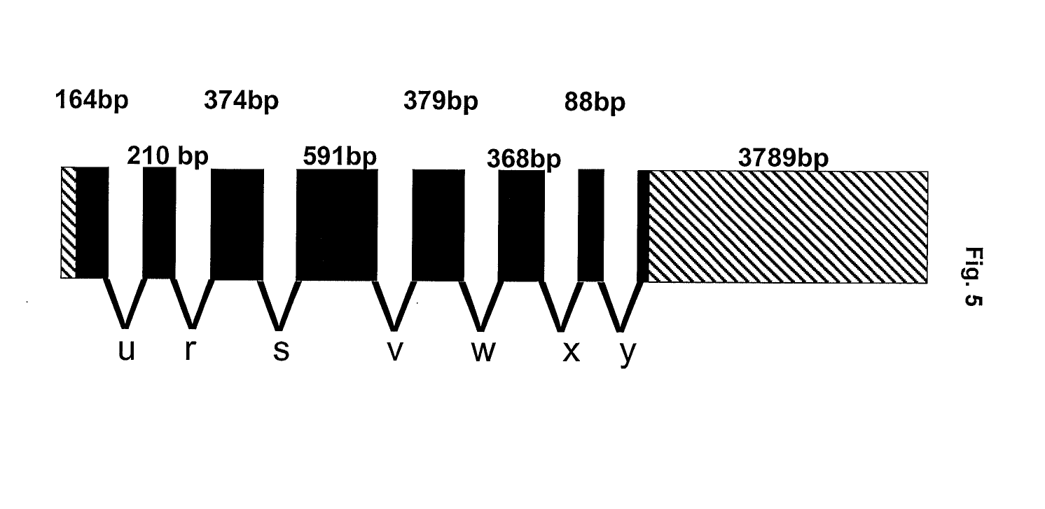

In order to check the resulting sequence from the LLcDNA cloning, two fragments were amplified by PCR (SEQ ID NO: 15 and SEQ ID NO: 16 or SEQ ID NO: 6 and SEQ ID NO: 17) and used to screen Lambda gt10 cDNA phage libraries. Positive plaques were isolated and amplified by PCR using gt10-specific primers (SEQ ID NO: 18 and SEQ ID NO: 19). Subsequent sequencing and alignment with the sequences led to the assumption that this is a differentially spliced product. The splice donor, acceptor and lariat sequence were found subsequently. Using PCR by a suitable combination of primers, searches were carried out in various cell lines for differential splice products; in all the cell lines screened, only one product was found and led,to the gene structure shown in FIG. 5. The cDNA shown has an open reading frame (ORF) which codes for a potential protein 749 amino acids long. The translation initiation site at position 215 corresponds to approximately 75% of a Kozak consensus sequence. The results obtained in this experiment led to the transcription initiation site being determined exactly by primer extension in another experiment (Example 6b), in order to be certain that the 5′ end found is actually the 5′ end of B345. The amino acid sequence of B345 derived from the cDNA (SEQ ID NO: 1) obtained in this cloning test is shown in SEQ ID NO: 2.

b) Second cloning test; determining the 5′ and promotor region of B345

Using the Promotor Finder DNA Walking Kit (Clontech) and subsequent primer extension reaction, the 5′ region and the promoter region and also the exact transcription initiation site were determined. The 5′ region was amplified using a genomic DNA library produced by Clontech with B345 specific primers (SEQ ID NO:38 or nested SEQ ID NO: 39) and Adaptor Primer in the kit. To determine the exact start of transcription, the Primer Extension reaction was carried out. To do this, the primer SEQ ID NO: 40 was labelled with 10 U of T4 Polynukleotide Kinase (Promega) and 3 μl of [γ-³²P]ATP (3000 Ci/mmol) at the 5′ end according to standard protocols (Sambrook et al., 1989). The labelled oligonucleotide was purified by precipitation. For the primer extension reaction, 10,000 cpm of oligonucleotide were used in a total volume of 20 μl to 25 μg of Total RNA of the Colo 205 cell line (ATCC:CCL-222).

The RNA of the cell line was reverse transcribed with the radioactively labelled primer and applied to a 10% polyacryamide gel. To determine the exact band length a PCR fragment of nt 1000-nt 1362 was sequenced with ³⁵S labelled nucleotides and also applied. The fragment of 209 nucleotides resulting from the elongation of the reverse primer fixes the start of transcription precisely at position 201. In this way the B345 sequence obtained in Example 6a was extended in the 5′ region and a new start codon was located at position 283. By repeated sequencing even in the 3′ region, an additional base, compared with the sequence shown in SEQ ID NO: 1, was found at position 2430, leading to a shift in the reading frame and thus displaces the stop codon from position 2729 to 2791. The cDNA (SEQ ID NO: 3) obtained in this experiment has an open reading frame which codes for a potential protein 836 amino acids long (SEQ ID NO: 4).

### EXAMPLE 7

Bioinformatic analysis of the function of B345

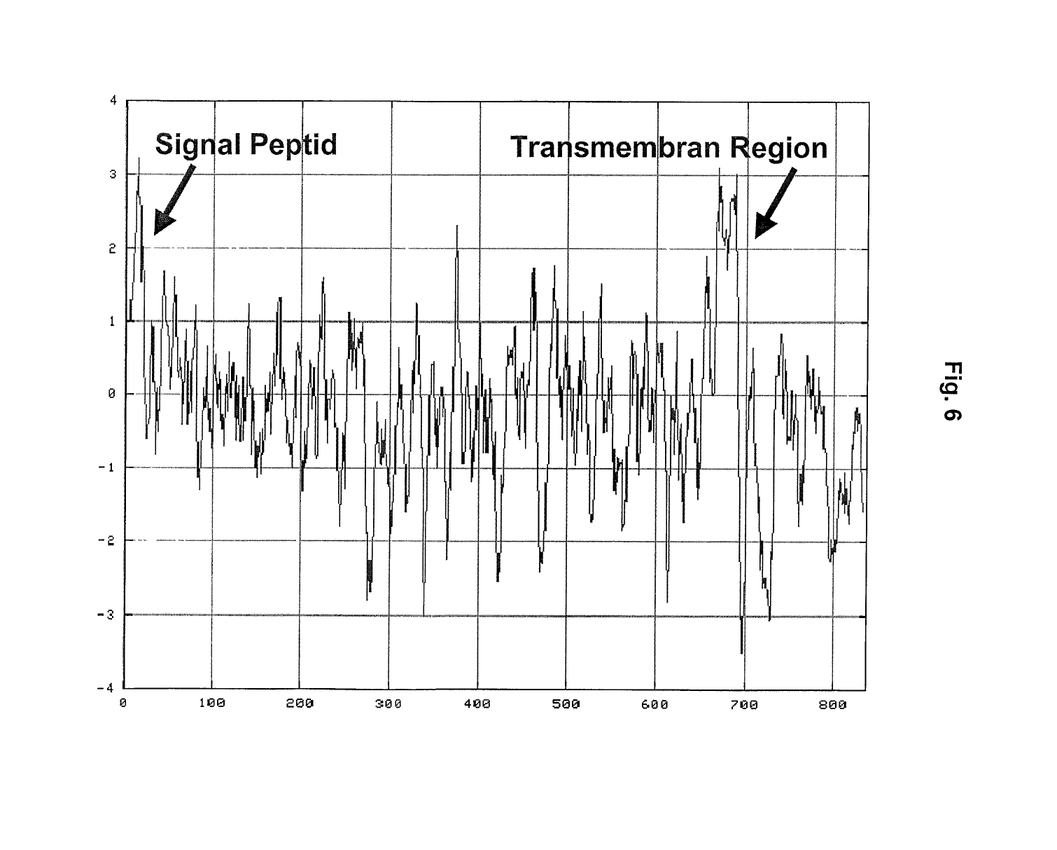

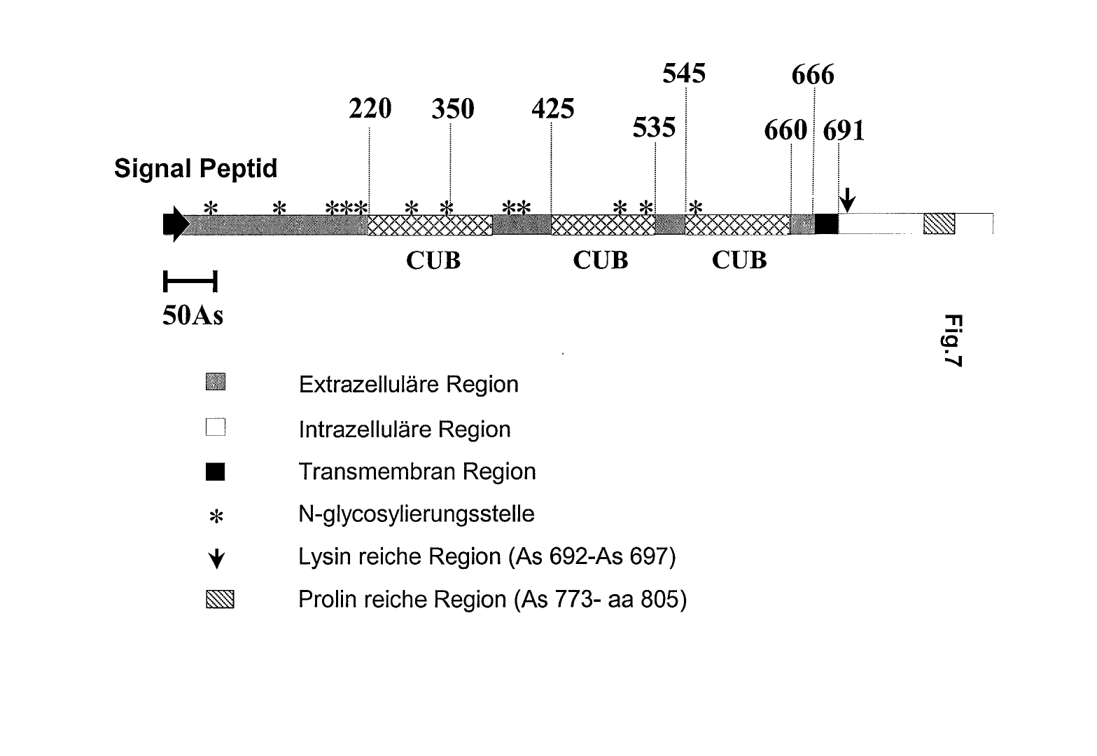

The resulting primary amino acid sequence of B345 is shown in SEQ ID NO: 4. Analysis of the hydrophilicity plot of the amino acid sequence using the method of Kyte and Doolittle (1982) shows that the B345 protein has two characteristic hydrophobic domains (amino acids pos. 1-29 and 666-691), which represent a signal peptide and a helical transmembrane domain (FIG. 6). This polarised structure indicates that B345 is an integral membrane protein. The transmembrane helix connects an extracellular section about 666 amino acids long and a short intracellular section (145 amino acids) (cf. FIG. 7).

The extracellular domain also shows clear signs of the existence of a CUB domain at positions 220-350 and signs of 2 possible other CUB domains in the region of amino acids 425-660. CUB domains occur at various proteins, generally regulated during the development of the embryo. In addition, CUB domains can sometimes also be found at EGF (Epidermal Growth Factor)-like domains. A recent publication (Gerstein et al., 2000) demonstrates that proteins containing CUB domains are the most markedly differentially regulated proteins in C. elegans. Since genes which play a key role in embryo development also have analogous functions in cancer, it can be assumed that the overexpression of B345 in cells brings about a change in the properties of the substrate adhesion or the extracellular matrix. The protein also has 12 potential N-glycosylation sites which can be found in the predicted extracellular domain, which concurs with the predicted orientation of the protein.

With a BLAST hit (E-value: 5.8×10⁻²) for the region of amino acids 235 to 282 of B345 it was possible to identify a complement-activating component of the reactive factor (RARF) from mus musculus. The alignment is located within the CUB domain 1 of B345.

The CUB domains 2 and 3 (section 425-535 and 545-660) exhibit marginal homology with the human and fugu procollagen C proteinase enhancer protein (PCOLCE). These regions occur in the part of PCOLCE which contains a CUB domain tandem repeat (E-values: 0.5 (human) and 2.7 (fugu)). CUB domains sometimes occur in repeats.

Presumably, the B345 protein forms a β-sheet secondary structure, as it is known that CUB domains fold into a β sandwich.

The intracellular domain (section 691-836) has no significant homologies. However, the total C-terminus aligned with an EST (AW063026) of human ovarian cancer cells (82% identity over 124 amino acids).

### EXAMPLE 8

Determining the precise genetic structure of B345

First of all, Bac clones were sought in public data banks (BLAST search) which contain the B345 gene. The Bac clones Ac068625 and Ac010170 contained a major part of the gene. With intron-spanning primers, splice acceptor and donor sequences were sought in Colo 205 cDNA and genomic DNA was sought as a template. The PCR procedure was carried out as follows: 1× 95° C. for 2 minutes, 35× 95° C. for 15 seconds, 68° C. for 3 minutes and then kept at 4° C. The PCR was analysed on a 1.2% agarose gel and the lengths of the PCR products of the 2 templates were compared with identical primer combinations. It was found that B345 consists of 8 exons, separated by 7 introns (FIG. 5).

The chromosomal location of the gene was determined by fluorescence-in situ-hybridisation (FISH). The human, digoxigenin-labelled B345 probe together with the biotin-labelled probe of B47a2 (Knight et al., 1997), which is found at the sub-telomeric region of the 3p arm of the chromosome, was hybridised with metaphase chromosomes of two “normal” individuals (Lichter et al., 1988). The hybridised digoxygenin probe was detected by anti-sheep-Dig (Boehringer Mannheim FRG) and rabbit anti-sheep FITC-labelled antibodies. The biotin-labelled probe, on the other hand, was made visible with mouse anti-biotin and rabbit-anti-mouse (TRITC) and by subsequently staining with DAPI. The FISH results show that a majority of the metaphases have clear signals on one or both chromatides of chromosome 3 in the region p21-p23. The co-location of the B47a2 (TRITC) probe on the same chromosomal arm served to confirm the position.

TABLE 1A

| Tissue | expression |
|---|---|
| PBL | − |
| lung | ++ |
| placenta | + |
| small intestine | + |
| liver | − |
| kidney | ++ |
| spleen | − |
| thymus | − |
| colon | + |
| skeletal muscle | − |
| heart | − |
| brain | − |

TABLE 1B

| cell line | expression |
|---|---|
| promyelocytic leukaemia HL60 | − |
| HELA cells S3 | − |
| chronic myelogenic leukaemia K-562 | + |
| lymphoblastic leukaemia MOLT-4 | − |
| Burkitt's lymphoma (Raji) | − |
| colon adenocarcinoma SW480 | +++ |
| lung adenocarcinoma A549 | + |
| melanoma G361 | − |

TABLE 1C

| Tissue | Expression |
|---|---|
| Oesophagus Tumor | (+) |
| Normal Oesophagus | (+) |
| Stomach Tumor | − |
| Normal Stomach | + |
| Colon Tumor | +++ |
| Normal Colon | ++ |
| Rectum Tumor | + |
| Normal Rectum | (+) |

TABLE 2A

| | Expression B345/ | Expression B345 |
|---|---|---|
| Tissue | Actin | Tubulin |
| Lung Adenocarcinoma | + | + |
| Lung Adenocarcinoma | + | + |
| Normal Lung | − bis (+) | (+) |
| Colon Adenocarcinoma | ++ | ++ |
| Colon Adenocarcinoma | +++ | +++ |
| Normal Colon | − bis (+) | + |
| Breast IDC | + | + |
| Breast | − | − |
| Hodgkin's Lymphoma | − | − |
| Spleen | − | − |
| Testis | − | − |

TABLE 2B

| Cell lines and Tissues | Expression B345/GAPDH |
|---|---|
| Colon Adenocarcinoma SW480 | + |
| Normal Colon (Clontech) | (+) |
| Normal Colon (Invitrogen) | (+) |
| Lung Adenocarcinoma A549 | (+) |
| Colon Adenocarcinoma Colo 205 | +++ |
| PG 102142 Tumor (Colon Ac.) | +++ |
| PG 21900 Tumor (Colon Ac.) | ++ |
| PG 7066 Tumor (Colon Ac.) | +++ |
| PG 32389 Tumor (Colon Ac.) | ++ |

### Bibliography

Abe, K., Rapid isolation of desired sequences from lone linker PCR amplified CDNA mixture: application to identification and recovery of expressed sequences in cloned genomic DNA. Mamm. Genome 2,252-259

Altschul, S. F., Madden, T. L., Schäffer, A. A., Zhang, J., Zhang, Z., Miller, W. and Lipman, D. J. Gapped BLAST and PSI-BLAST: a new generation of protein database search programs. Nucleic Acids Res. 25, 3389-3402 (1997).

Bateman, A., Birney, E., Durbin, R., Eddy, S. R., Howe, K. L. and Sonnhammer, E. L. The Pfam Protein Families Database. Nucleic Acids Res. 28, 263-266 (2000).

Bird, A. P. (1986). CpG-rich islands and the function of DNA methylation. Nature 321:209-213.

Bork, P. et al. (1993). The CUB domain. A widespread module in developmentally regulated proteins. J.Mol. Biol. 231: 539-545

Boulianne, G. L., et al., (1984), Nature 312: 643-646

Böhm et al., A.J. of Pathology 151,1:63-67, 1997

Brüggemann, M. und Neuberger, M.S., (1996), Immunol. Today 17: 391-397

Cuff, J. A., Clamp, M. E., Siddiqui, A. S., Finlay, M. and Barton, G. J. Jpred: a consensus secondary structure prediction server. Bioinformatics 14, 892-893 (1998).

Diatchenko, L., Lau, Y. F., Campbell, A. P., Chenchik, A., Moqadam, F., Huang, B., Lukyanov, S., Lukyanov, K., Gurskaya, N., Sverdlov, E. D., and Siebert, P. D. (1996), Proc. Natl. Acad. Sci. U. S. A. 93, 6025-6030.

Fields S, Song O (1989) A novel genetic system to detect protein-protein interactions. Nature 20; 340(6230):245-6

Gaudi C, Kremer F, Angevin E, Scott V, Triebel F (1999), J Immunol 162:1730-1738

Gerstein, M. and Jansen, R. (2000). The current excitement in bioinformatics-analysis of whole-genome expression data: how does it relate to protein structure and function. Curr. Opin. Struct. Biol. 10:574-584.

Graziano, R. F., et al., (1995), J. Immunol. 155: 4996-5002

Griffiths, A.D., et al., (1994), EMBO J. 13: 3245-3260

Grosveld, F. and Kollias, G. Transgenic Animals, Academic Press (1992)

Hamburger, A. W. and Salmon, S. E. (1977). Primary bioassay of human tumor stem cells. Science. 197 (4302):461-463.

Hesketh, R., (1995), The oncogene, Academic Press

Higgins, D. G., Thompson, J. D. and Gibson, T. J. Using CLUSTAL for Multiple Sequence Alignments. Methods Enzymol. 266, 383-402 (1996).

Hogan K T, Eisinger D P, Cupp S B C, Lekstrom K J, Deacon D D, Shabanowitz J, Hunt D F, Engelhard V H, Slingluff C L, Ross M M (1998), Cancer Res 58:5144-5150

Hofmann, K., Buchner, P., Falquet, L. and Bairoch, A. The PROSITE database, its status in 1999. Nucleic Acids Res. 27, 215-219 (1999).

Hubank, M. and Schatz, D.G., (1994), Nucleic. Acids. Res. 22, 5640-5648.

Jakobovits, A., (1995), Curr. Opin. Biotechnol. 6: 561-566

Kasten, M. B., (1997), Genetic Instability and Tumorigenesis, Springer Verlag

Knight, S. J., Horsley, S. W., Regan, R., Lawrie, N.

M., Maher, E. J., Cardy, D. L., Flint, J., and Kearney, L. (1997). Development and clinical application of an innovative fluorescence in situ hybridization technique which detects submicroscopic rearrangements involving telomeres. Eur. J. Hum. Genet. 5:1-8.

Köhler, G. und Milstein, C. (1975), Nature 265, 495-497

Kozak, M (1987), An analysis of 5′ noncoding sequences from 99 vertebrates messenger RNA's. Nuc.Ac.Res. Vol.15: 8125-8147

Kruif, J., et al., (1995), Proc. Natl. Acad. Sci. USA 92: 3938-3942

Kyte, J and Doolittle, R F (1982), J. Mol. Biol. 157: 105-132

Liaw, L., Skinner, M. P., Raines, E. W., Ross, R., Cheresh, D. A., Schwartz, S. M., and Giachelli, C. M. (1995). The adhesive and migratory effects of osteopontin are mediated via distinct cell surface integrins. Role of alpha v beta 3 in smooth muscle cell migration to osteopontin in vitro. J.Clin.Invest. 95 (2):713-724.

Lichter, P., Cremer, T., Borden, J., Manuelidis, L., and Ward, D. C. (1988). Delineation of individual human chromosomes in metaphase and interphase cells by in situ suppression hybridization using recombinant DNA libraries. Hum. Genet. 80:224-234.Mandruzzato S. Brasseur F, Andry G, Boon T, van der Bruggen P (1997), J Exp Med 186:785-793

McGuinnes, B. T., et al., (1996), Nature Biotechnol. 14, 1149

Neuberger, M. S., et al., (1984), Nature 312: 604-608

Pearson, W. R. and Lipman, D. J. Improved tools for biological sequence comparison. Proc. Natl. Acad. Sci. USA 85, 2444-2448 (1988).

Pusztal et al., 1996, cell proliferation in cancer, Oxford medical publications

Rauscher, F. J. et al., (1997), Chromosomal translocations and oncogenic transcription factors, Springer Verlag

Riechmann, L., et al., (1988), Nature 332: 323-327

Sambrook, J., Fritsch, E. F., and Maniatis, T. (1989). “Molecular Cloning: A laboratory Manual” 2nd ed., Cold Spring Laboratory Press, Cold Spring Harbor, N.Y.

Schultz, J., Copley, R. R., Doerks, T., Ponting, C. P. and Bork, P. SMART: A Web-based tool for the study of genetically mobile domains. Nucleic Acids Res. 28, 231-234 (2000).

Toes, R. E., Hoeben, R. C., Van der Voort, E., Ressing, M. E., Van-der-Eb, A. J. Melief, C. J. M., and Offringa, R. (1997), Proc. Natl. Acad. Sci. U.S.A. 94 (26): 14660-14665

Velculescu, V E, Zhang, L, Vogelstein, B, and Kinzler, K W (1995), Science 270: 484-487

Winter, G., et al., (1994), Annu. Rev. Immunol. 12, 433-455

Woelfel, T, Schneider, J, Zum Buschenfelde, Meyer, K H, Rammensee, H G, Rotzschke, O, and Falk, K (1994), Int. J. Cancer 57: 413-418

Wu, T. C., Guarnieri, F. G., Staveley-O'Carroll, K. F., Viscidi, R. P., Levitsky, H. I., Hedrick, L., Cho, K. R., August, J. T., and Pardoll, D. M. (1995), Proc. Natl. Acad. Sci. U.S.A. 92(25): 11671-11675.

1. Tumour-associated antigen designated B345, characterised in that it has the amino acid sequence defined in SEQ ID NO: 4 or contains this as part of its sequence, or a fragment thereof.

2. Isolated DNA molecule, coding for the tumour-associated antigen defined in claim 1 or for fragments thereof.

3. DNA molecule according to claim 2, characterised in that it is a polynucleotide with the sequence shown in SEQ ID NO: 3 or contains this sequence, or in that it is or contains a polynucleotide which hybridises under stringent conditions with a polynucleotide of the sequence shown in SEQ ID NO: 3, or a fragment thereof.

4. Recombinant DNA molecule, containing a DNA molecule according to claim 2 or 3.

5. Pharmaceutical composition for cancer immunotherapy, containing as active ingredient the tumour-associated antigen designated B345 defined in claim 1 or one or more fragments thereof.

6. Pharmaceutical composition for cancer immunotherapy, containing as active ingredient a DNA molecule according to one of claims 2 to 4.

7. Antibody against the polypeptide defined in claim 1.

8. Antibody according to claim 7, characterised in that it is monoclonal.

9. Antibody according to claim 7 or 8 for the treatment and diagnosis of cancers associated with the expression of B345.


SEQUENCE LISTING (40 sequences)

SEQ ID NO: 1 - DNA, 5897 bp, Homo sapiens
  misc_feature (5348)...(5348): n is any nucleotide of a, t, g or c
  misc_feature (5360)...(5360): n is any nucleotide of a, t, g or c
  misc_feature (5425)...(5425): n is any nucleotide of a, t, g or c
  misc_feature (5438)...(5438): n is any nucleotide of a, t, g or c
  5′UTR (1)..(214)
  CDS (215)..(2464)
  3′UTR (2465)..(5897)
cttgagatattagaattcgcgactcctgaactgcggggtctctatcgcactgctaggggttctgctgctgggtgcggcgcgcctgccgcgcggggcagaagcttttgagattgctctgccacgagaaagcaacattacagttctcataaagctggggaccccgactctgctggcaaaaccctgttacatcgtcatttctaaaagacatataaccatgttgtccatcaagtctggagaaagaatagtctttacctttagctgccagagtcctgagaatcactttgtcatagagatccagaaaaatattgactgtatgtcaggcccatgtccttttggggaggttcagcttcagccctcgacatcgttgttgcctaccctcaacagaactttcatctgggatgtcaaagctcataagagcatcggtttagagctgcagttttccatccctcgcctgaggcagatcggtccgggtgagagctgcccagacggagtcactcactccatcagcggccgaatcgatgccaccgtggtcaggatcggaaccttctgcagcaatggcactgtgtcccggatcaagatgcaagaaggagtgaaaatggccttacacctcccatggttccaccccagaaatgtctccggcttcagcattgcaaaccgctcatctataaaacgtctgtgcatcatcgagtctgtgtttgagggtgaaggctcagcaaccctgatgtctgccaactacccagaaggcttccctgaggatgagctcatgacgtggcagtttgtcgttcctgcacacctgcgggccagcgtctccttcctcaacttcaacctctccaactgtgagaggaaggaggagcgggttgaatactacatcccgggctccaccaccaaccccgaggtgttcaagctggaggacaagcagcctgggaacatggcggggaacttcaacctctctctgcaaggctgtgaccaagatgcccaaagtccagggatcctccggctgcagttccaagttttggtccaacatccacaaaatgaaagcaataaaatctacgtggttgacttgagtaatgagcgagccatgtcactcaccatcgagccacggcccgtcaaacagagccgcaagtttgtccctggctgtttcgtgtgtctagaatctcggacctgcagtagcaacctcaccctgacatctggctccaaacacaaaatctccttcctttgtgatgatctgacacgtctgtggatgaatgtggaaaaaaccataagctgcacagaccaccggtactgccaaaggaaatcctactcactccaggtgcccagtgacatcctccacctgcctgtggagctgcatgacttctcctggaagctgctggtgcccaaggacaggctcagcctggtgctggtgccagcccagaagctgcagcagcatacacacgagaagccctgcaacaccagcttcagctacctcgtggccagtgccatacccagccaggacctgtacttcggctccttctgcccgggaggctctatcaagcagatccaggtgaagcagaacatctcggtgacccttcgcacctttgcccccagcttccaacaagaggcctccaggcagggtctgacggtgtcctttataccttatttcaaagaggaaggcgttttcacggtgacccctgacacaaaaagcaaggtctacctgaggacccccaactgggaccggggcctgccatccctcacctctgtgtcctggaacatcagcgtgcccagagaccaggtggcctgcctgactttctttaaggagcggagcggcgtggtctgccagacagggcgcgcattcatgatcatccaggagcagcggacccgggctgaggagatcttcagcctggacgaggatgtgctccccaagccaagcttccaccatcacagcttctgggtcaacatctctaactgcagccccacgagcggcaagcagctagacctgctcttctcggtgacacttaccccaaggactgtggacttgactgtcatcctcatcgcagcggtgggaggtggagtcttactgctgtctgccctcgggctcatcatttgctgtgtgaaaaagaagaaaaagaagacaaacaagggccccgctgtgggtatctacaatggcaacatcaatactgagatgccaggcagccaaaaaagtttcagaaagggcgaaaggacaatgactcccatgtgtatgcagtcatcgaggacaccatggtatatgggcatctgctacaggattccagcggctccttcctgcagccagaggtggacacctaccggccgttccagggcaccatgggggtctgtcctccctccccacccaccatatgctccagggccccaactgcaaagttggccactgaggagccacctcctcgctcccctcctgagtctgagagtgaaccgtacaccttctcccatcccaacaatggggatgtaagcagcaaggacacagacattcccttactgaacactcaggagcccatggagccagcagaataacttgatccattccagacgctttgctgagtttcataaagcagggcactgagacacccgtccgtgttcctaaccagaaatcctaaagaagaggaattatacagaaggaacagcaggaggttttcctggacaccgccaacttcacattgctcagtggactcattctaagggcaagacattgaaaatgatgaattccaatctggatacagtcatgacagctcatgtgctcctcaacttaggctgtgcggttagccagcctgtaatgagaggagagaggcctgagtcacctagcatagggttgcagcaagccctggattcagagtgttaaacagaggcttgccctcttcaggacaacagttccaattccaaggagcctacctgaggtccctactctcactggggtccccaggatgaaaacgacaatgtgcctttttattattatttatttggtggtcctgtgttatttaagagatcaaatgtataaccacctagctcttttcacctgacttagtaataactcatactaactggtttggatgcctgggttgtgacttctactgaccgctagataaacgtgtgcctgtcccccaggtggtgggaataatttacaatctgtccaaccagaaaagaatgtgtgtgtttgagcagcattgacacatatctgctttgataagagacttcctgattctctaggtcggttcgtggttatcccattgtggaaattcatcttgaatcccattgtcctatagtcctagcaataagagaaatttcctcaagtttccatgtgcggttctcctagctgcagcaatactttgacatttaaagagaaatttagagaatattctcatcctctaaaaatgtttaaatatataccaaacagtggccccctgcattagttttctgttgccactgcaacccattacttggtagcttaaaaacaacacattagcttatagtcctggggatcagaattccaaaatggatgtccctgaatgaaaatcaaggtgtcagcagagctgtgctccttctgaaggctctagggagaagccggttccttgccatttcaagcttctagaggctggctgcattcccaggctccagtggctggtcaagcttttctcacatggcatcactgtgacactggccctcccacttccctctttgacttacaaagcccaccaggaagatccaggataatctctccatctaaagatccttcatcatcctggaagagccttttgccatgcaagacaacatagccacaggtggggattaggaccaggacatctttggggtgctgttattctgcctaccacaccttcctgccacbgactcccacaggagaggctacaaaatgatctggcgcacagggatgttttgtttagcttgcggactctaacacttaaaaaaaccccagatcagaagatctggccatgctggggctcacattctcacctagcaacaactggctggagctgggcaccagctctgcctttagaaggggtgtccacttcaccaggtcaccacagcccacactacgccctatcacttcccacaatgaggctaagtgtttgtttctactgatcaatgcccctgcaggttgcatttattgtaatgaaaaagaaagactgggattaatctctaatcaggtgagtagaccatgagaccaatgtgtgctcacattaccctttttcttttttttctttttctttttctttttttttttaatgtgagacaggatctcattctgttgcctaggctggagtgcagtggcgcaatctcggctcactgcaacctctgcctcctgggctcaagcaattctcccacctcagcctcccaaatagctgggatcactggcacaaaccaccatgcccagctaattttgtattttttgtagagacagggtttcaccatgttgcccaggctggtctcaacctcctgggctcaagcaatcctcctgcctcggcctcccaaagtgctgggattacagatgtgagccaccgcatccagccccacaccctcatttataccaattacctgcccagtaactgtggacttttgcttcctcacccctgctctgatctggaaggagagggattatgttatagcttgtcagcacagtcccaagttcaatatttctgcggcaaaaacttccttcaaaaaataaatgtacttcattgtattcaatgaattcaccttggaaatgcaccgcctcaacttgttcacatggcataaatgaaaggaattttatagtctcctaaatggcgtgtactgcaagacctcttgaacactttccagaggataggatatttaagtcatgcccttggcgttgcctatggcacctttcccttctgaaagtctggttcctgcccagtgacccttggccttgtgagccgagatgctgaccctgcataaagggccaaaggagggctgcggcttccttccctcactgaagagcccttatttgaattcactgtgtggagccctagccctccattctcgacattccccaacctcccagccccttccaagcaggactaggtgccctgcattccacccaaggtgggattggccttccttaggctggctacttgtcaccatcaccgacatcactgttgcctgcaaggacaccacgtggccattttccttcaactgagggctcaaaactcctggacaagttgctggctcctgagaccagtatttcctggagmtgtgcctcagtgaaggggcccagcctgaggaaccctggctcttttctttaaagcccaggccccacttacataaaacatttcagggtcactggaaacagtgaagtgccatttgtngaagcctactgnatgccagcccactgctcatccacgtggtatgccatgcctacgaggaaggccagcgcatgcaggantggtctctaatgntgtggtcattgcacagaagggaaaggtctcaaggaagagtcaactgggacaagcacaagcccaccggacatggccttggtaaaggttagcagactggtgtgtgtggatctgcagtgcttcactggaaataatttattcattgcagatactttttaggtggcattttattcatttcctgtgctttaaataaacaaatgtaccaaaaaacaagtatcaagctgtttaagtgcttcggctacttgtcccctggttcagtagaggccccggtttcccagttgttgactgtgacaggctcagcatgggctcagcagatgctgtcttaatttgtggatgatacagaaagccaggctttgggatacaagttctttcctcttcatttgatgccgtgcactgtgtgaagcagatgtttttgtccggaaataaaaataatagtcttggagtctcgccaaaaaaaaaaag

SEQ ID NO: 2 - protein, 749 aa, Homo sapiens
MLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNGNINTEMPGSQKSFRKGERTMTPMCMQSSRTPWYMGICYRIPAAPSCSQRWTPTGRSRAPWGSVLPPHPPYAPGPQLQSWPLRSHLLAPLLSLRVNRTPSPIPTMGM

SEQ ID NO: 3 - DNA, 6163 bp, Homo sapiens
  5′UTR (1)..(282)
  GC_signal (147)..(157)
  misc_feature (201)..(209): cap signal; Transcription start
  3′UTR (2794)..(6163)
  3′UTR (2794)..(6163)
  CDS (283)..(2793)
ccaacgccgcaatggggagtagtagggacccagcaacccggtgccgggagccctgcaccctgggagggagaggcggtcgctgaggcaggaagaggaggaggagagagaggagggacgcaccgggtcagctcgcgatcctgctgcgcagggcggggctcgggccggtccgcccgcgcgcaggtgagtgagccagggcggagcgcagctgcgccgggcttgggcgcctggggccgccgctccccaccgtcgttttccccaccgaggccgaggcgtcccggagtcatggccggcctgaactgcggggtctctatcgcactgctaggggttctgctgctgggtgcggcgcgcctgccgcgcggggcagaagcttttgagattgctctgccacgagaaagcaacattacagttctcataaagctggggaccccgactctgctggcaaaaccctgttacatcgtcatttctaaaagacatataaccatgttgtccatcaagtctggagaaagaatagtctttacctttagctgccagagtcctgagaatcactttgtcatagagatccagaaaaatattgactgtatgtcaggcccatgtccttttggggaggttcagcttcagccctcgacatcgttgttgcctaccctcaacagaactttcatctgggatgtcaaagctcataagagcatcggtttagagctgcagttttccatccctcgcctgaggcagatcggtccgggtgagagctgcccagacggagtcactcactccatcagcggccgaatcgatgccaccgtggtcaggatcggaaccttctgcagcaatggcactgtgtcccggatcaagatgcaagaaggagtgaaaatggccttacacctcccatggttccaccccagaaatgtctccggcttcagcattgcaaaccgctcatctataaaacgtctgtgcatcatcgagtctgtgtttgagggtgaaggctcagcaaccctgatgtctgccaactacccagaaggcttccctgaggatgagctcatgacgtggcagtttgtcgttcctgcacacctgcgggccagcgtctccttcctcaacttcaacctctccaactgtgagaggaaggaggagcgggttgaatactacatcccgggctccaccaccaaccccgaggtgttcaagctggaggacaagcagcctgggaacatggcggggaacttcaacctctctctgcaaggctgtgaccaagatgcccaaagtccagggatcctccggctgcagttccaagttttggtccaacatccacaaaatgaaagcaataaaatctacgtggttgacttgagtaatgagcgagccatgtcactcaccatcgagccacggcccgtcaaacagagccgcaagtttgtccctggctgtttcgtgtgtctagaatctcggacctgcagtagcaacctcaccctgacatctggctccaaacacaaaatctccttcctttgtgatgatctgacacgtctgtggatgaatgtggaaaaaaccataagctgcacagaccaccggtactgccaaaggaaatcctactcactccaggtgcccagtgacatcctccacctgcctgtggagctgcatgacttctcctggaagctgctggtgcccaaggacaggctcagcctggtgctggtgccagcccagaagctgcagcagcatacacacgagaagccctgcaacaccagcttcagctacctcgtggccagtgccatacccagccaggacctgtacttcggctccttctgcccgggaggctctatcaagcagatccaggtgaagcagaacatctcggtgacccttcgcacctttgcccccagcttccaacaagaggcctccaggcagggtctgacggtgtcctttataccttatttcaaagaggaaggcgttttcacggtgacccctgacacaaaaagcaaggtctacctgaggacccccaactgggaccggggcctgccatccctcacctctgtgtcctggaacatcagcgtgcccagagaccaggtggcctgcctgactttctttaaggagcggagcggcgtggtctgccagacagggcgcgcattcatgatcatccaggagcagcggacccgggctgaggagatcttcagcctggacgaggatgtgctccccaagccaagcttccaccatcacagcttctgggtcaacatctctaactgcagccccacgagcggcaagcagctagacctgctcttctcggtgacacttaccccaaggactgtggacttgactgtcatcctcatcgcagcggtgggaggtggagtcttactgctgtctgccctcgggctcatcatttgctgtgtgaaaaagaagaaaaagaagacaaacaagggccccgctgtgggtatctacaatggcaacatcaatactgagatgccgaggcagccaaaaaagtttcagaaagggcgaaaggacaatgactcccatgtgtatgcagtcatcgaggacaccatggtatatgggcatctgctacaggattccagcggctccttcctgcagccagaggtggacacctaccggccgttccagggcaccatgggggtctgtcctccctccccacccaccatatgctccagggccccaactgcaaagttggccactgaggagccacctcctcgctcccctcctgagtctgagagtgaaccgtacaccttctcccatcccaacaatggggatgtaagcagcaaggacacagacattcccttactgaacactcaggagcccatggagccagcagaataacttgatccattccagacgctttgctgagtttcataaagcagggcactgagacacccgtccgtgttcctaaccagaaatcctaaagaagaggaattatacagaaggaacagcaggaggttttcctggacaccgccaacttcacattgctcagtggactcattctaagggcaagacattgaaaatgatgaattccaatctggatacagtcatgacagctcatgtgctcctcaacttaggctgtgcggttagccagcctgtaatgagaggagagaggcctgagtcacctagcatagggttgcagcaagccctggattcagagtgttaaacagaggcttgccctcttcaggacaacagttccaattccaaggagcctacctgaggtccctactctcactggggtccccaggatgaaaacgacaatgtgcctttttattattatttatttggtggtcctgtgttatttaagagatcaaatgtataaccacctagctcttttcacctgacttagtaataactcatactaactggtttggatgcctgggttgtgacttctactgaccgctagataaacgtgtgcctgtcccccaggtggtgggaataatttacaatctgtccaaccagaaaagaatgtgtgtgtttgagcagcattgacacatatctgctttgataagagacttcctgattctctaggtcggttcgtggttatcccattgtggaaattcatcttgaatcccattgtcctatagtcctagcaataagagaaatttcctcaagtttccatgtgcggttctcctagctgcagcaatactttgacatttaaagagaaatttagagaatattctcatcctctaaaaatgtttaaatatataccaaacagtggccccctgcattagttttctgttgccactgcaacccattacttggtagcttaaaaacaacacattagcttatagtcctggggatcagaattccaaaatggatgtccctgaatgaaaatcaaggtgtcagcagagctgtgctccttctgaaggctctagggagaagccggttccttgccatttcaagcttctagaggctggctgcattcccaggctccagtggctggtcaagcttttctcacatggcatcactgtgacactggccctcccacttccctctttgacttacaaagcccaccaggaagatccaggataatctctccatctaaagatccttcatcatcctggaagagccttttgccatgcaagacaacatagccacaggtggggattaggaccaggacatctttggggtgctgttattctgcctaccacaccttcctgccactgactcccacaggagaggctacaaaatgatctggcgcacagggatgttttgtttagcttgcggactctaacacttaaaaaaaccccagatcagaagatctggccatgctggggctcacattctcacctagcaacaactggctggagctgggcaccagctctgcctttagaaggggtgtccacttcaccaggtcaccacagcccacactacgccctatcacttcccacaatgaggctaagtgtttgtttctactgatcaatgcccctgcaggttgcatttattgtaatgaaaaagaaagactgggattaatctctaatcaggtgagtagaccatgagaccaatgtgtgctcacattaccctttttcttttttttctttttctttttctttttttttttaatgtgagacaggatctcattctgttgcctaggctggagtgcagtggcgcaatctcggctcactgcaacctctgcctcctgggctcaagcaattctcccacctcagcctcccaaatagctgggatcactggcacaaaccaccatgcccagctaattttgtattttttgtagagacagggtttcaccatgttgcccaggctggtctcaacctcctgggctcaagcaatcctcctgcctcggcctcccaaagtgctgggattacagatgtgagccaccgcatccagccccacaccctcatttataccaattacctgcccagtaactgtggacttttgcttcctcacccctgctctgatctggaaggagagggattatgttatagcttgtcagcacagtcccaagttcaatatttctgcggcaaaaacttccttcaaaaaataaatgtacttcattgtattcaatgaattcaccttggaaatgcaccgcctcaacttgttcacatggcataaatgaaaggaattttatagtctcctaaatggcgtgtactgcaagacctcttgaacactttccagaggataggatatttaagtcatgcccttggcgttgcctatggcacctttcccttctgaaagtctggttcctgcccagtgacccttggccttgtgagccgagatgctgaccctgcataaagggccaaaggagggctgcggcttccttccctcactgaagagcccttatttgaattcactgtgtggagccctagccctccattctcgacattccccaacctcccagccccttccaagcaggactaggtgccctgcattccacccaaggtgggattggccttccttaggctggctacttgtcaccatcaccgacatcactgttgcctgcaaggacaccacgtggccattttccttcaactgagggctcaaaactcctggacaagttgctggctcctgagaccagtatttcctggagctgtgcctcagtgaaggggcccagcctgaggaaccctggctcttttctttaaagcccaggccccacttacataaaacatttcagggtcactggaaacagtgaagtgccatttgttgaagcctactgcatgccagcccactgctcatccacgtggtctgccatgcctacgaggaaggccagcgcatgcaggactggtctctaatgctgtggtcattgcacagaagggaaaggtctcaaggaagagtcaactgggacaagcacaagcccaccggacatggccttggtaaaggttagcagactggtgtgtgtggatctgcagtgcttcactggaaataatttattcattgcagatactttttaggtggcattttattcatttcctgtgctttaaataaacaaatgtaccaaaaaacaagtatcaagctgtttaagtgcttcggctacttgtcccctggttcagtagaggccccggtttcccagttgttgactgtgacaggctcagcatgggctcagcagatgctgtcttaatttgtggatgatacagaaagccaggctttgggatacaagttctttcctcttcatttgatgccgtgcactgtgtgaagcagatgtttttgtccggaaataaaaataatagtcttggagtctcgccaaaaaaaaaaa

SEQ ID NO: 4 - protein, 836 aa, Homo sapiens [sequence omitted]

SEQ ID NO: 5 - DNA, 23 bp, Artificial sequence
  Description of the artificial sequence: Primer
accgcctcaacttgttcacatgg

SEQ ID NO: 6 - DNA, 26 bp, Artificial sequence
  Description of the artificial sequence: Primer
ctggtctcaggagccagcaacttgtc

SEQ ID NO: 7 - DNA, 25 bp, Artificial sequence
  Description of the artificial sequence: Primer
ctcatgacgtggcagtttgtcgttc

SEQ ID NO: 8 - DNA, 26 bp, Artificial sequence
  Description of the artificial sequence: Primer
ggctcgctcattactcaagtcaacca

SEQ ID NO: 9 - DNA, 36 bp, Artificial sequence
  Description of the artificial sequence: Primer
attcgcgactgatgatcgattttttttttttttttt

SEQ ID NO: 10 - DNA, 20 bp, Artificial sequence
  Description of the artificial sequence: Primer
attcgcgactgatgatcgat

SEQ ID NO: 11 - DNA, 20 bp, Artificial sequence
  Description of the artificial sequence: Primer
gagatattagaattctactc

SEQ ID NO: 12 - DNA, 17 bp, Artificial sequence
  Description of the artificial sequence: Primer
gagtagaattctaatat

SEQ ID NO: 13 - DNA, 22 bp, Artificial sequence
  Description of the artificial sequence: Primer
agtccatgtgaacaagttgagg

SEQ ID NO: 14 - DNA, 20 bp, Artificial sequence
  Description of the artificial sequence: Primer
aattctcccacctcagcctc

SEQ ID NO: 15 - DNA, 22 bp, Artificial sequence
  Description of the artificial sequence: Primer
aggatgaaaacgacaatgtgcc

SEQ ID NO: 16 - DNA, 21 bp, Artificial sequence
  Description of the artificial sequence: Primer
agaattgcttgagcccaggag

SEQ ID NO: 17 - DNA, 21 bp, Artificial sequence
  Description of the artificial sequence: Primer
caacttcacattgctcagtgg

SEQ ID NO: 18 - DNA, 25 bp, Artificial sequence
  Description of the artificial sequence: Primer
tgagcaagttcagcctggttaagtc

SEQ ID NO: 19 - DNA, 26 bp, Artificial sequence
  Description of the artificial sequence: Primer
caccgaatactcataaagaaggtccc

SEQ ID NO: 20 - DNA, 26 bp, Artificial sequence
  Description of the artificial sequence: Primer
tagacttcgagcaggagatggccact

SEQ ID NO: 21 - DNA, 20 bp, Artificial sequence
  Description of the artificial sequence: Primer
ccagccatgtacgtagccat

SEQ ID NO: 22 - DNA, 19 bp, Artificial sequence
  Description of the artificial sequence: Primer
ccaagaaggaaggctggaa

SEQ ID NO: 23 - DNA, 25 bp, Artificial sequence
  Description of the artificial sequence: Primer
ccatcaccatcttccaggagcgaga

SEQ ID NO: 24 - DNA, 19 bp, Artificial sequence
  Description of the artificial sequence: Primer
ccaagaaggaaggctggaa

SEQ ID NO: 25 - DNA, 20 bp, Artificial sequence
  Description of the artificial sequence: Primer
tgcaggaggcattgctgatg

SEQ ID NO: 26 - DNA, 19 bp, Artificial sequence
  Description of the artificial sequence: Primer
aaatcgtgcacttgcaggc

SEQ ID NO: 27 - DNA, 18 bp, Artificial sequence
  Description of the artificial sequence: Primer
ttgatgcgttccagctga

SEQ ID NO: 28 - DNA, 21 bp, Artificial sequence
  Description of the artificial sequence: Primer
ttgaattcactgtgtggagcc

SEQ ID NO: 29 - DNA, 19 bp, Artificial sequence
  Description of the artificial sequence: Primer
tgcaggcaacagtgatgtc

SEQ ID NO: 30 - DNA, 24 bp, Artificial sequence
  Description of the artificial sequence: Primer
attggccttccttaggctggctac

SEQ ID NO: 31 - DNA, 43 bp, Artificial sequence
  Description of the artificial sequence: Primer
tgtagcgtgaagacgacagaaagggcgtggtaccgagctcgag

SEQ ID NO: 32 - DNA, 22 bp, Artificial sequence
  Description of the artificial sequence: Primer
agggcgtggtaccgagctcgag

SEQ ID NO: 33 - DNA, 11 bp, Artificial sequence
  Description of the artificial sequence: Primer
ggctcgagctc

SEQ ID NO: 34 - DNA, 22 bp, Artificial sequence
  Description of the artificial sequence: Primer
ggccatgtccggtgggcttgtg

SEQ ID NO: 35 - DNA, 26 bp, Artificial sequence
  Description of the artificial sequence: Primer
ctcaaaactcctggacaagttgctgg

SEQ ID NO: 36 - DNA, 22 bp, Artificial sequence
  Description of the artificial sequence: Primer
aaggtgaaggtcggagtcaacg

SEQ ID NO: 37 - DNA, 24 bp, Artificial sequence
  Description of the artificial sequence: Primer
ggcagagatgatgacccttttggc

SEQ ID NO: 38 - DNA, 23 bp, Artificial sequence
  Description of the artificial sequence: Primer
agcagcagaacccctagcagtgc

SEQ ID NO: 39 - DNA, 26 bp, Artificial sequence
  Description of the artificial sequence: Primer
agaacccctagcagtgcgatagagac

SEQ ID NO: 40 - DNA, 27 bp, Artificial sequence
  Description of the artificial sequence: Primer
gaactgtaatgttgctttctcgtggca